(19) United States
(12) Patent Application Publication
(10) Pub. No.: US 2025/0011718 A1
(43) Pub. Date: Jan. 9, 2025

(54) Ex Vivo Generation of Immune Effector Cells from Apheresis Material Intermediates

(71) Applicant: ImmunityBio, Inc., Culver City, CA (US)

(72) Inventor: Patrick Soon-Shiong, Culver City, CA (US)

(73) Assignee: ImmunityBio, Inc., Culver City, CA (US)

(21) Appl. No.: 18/762,522

(22) Filed: Jul. 2, 2024

Related U.S. Application Data
(60) Provisional application No. 63/605,326, filed on Dec. 1, 2023.
(60) Provisional application No. 63/515,528, filed on Jul. 25, 2023.
(60) Provisional application No. 63/512,032, filed on Jul. 5, 2023.

(51) Int. Cl. C12N 5/0783; A61K 39/00

(52) U.S. Cl. CPC C12N 5/0636; A61K 39/4611; A61K 39/4613; A61K 39/464499; C12N 5/0646; C12N 2501/22; C12N 2501/2304

(57) ABSTRACT

Methods and compositions are provided herein for generating tumor targeted lymphocytes and/or tumor infiltrating lymphocytes and/or tumor targeted natural killer (NK) cells for use in the treatment of a cancer or an infectious disease. Also provided herein are methods of making and using the same.

This application claims the benefit of priority to U.S. Provisional Application Nos. 63/512,032 filed on Jul. 5, 2023; 63/515,528 filed on Jul. 25, 2023, and 63/605,326 filed on Dec. 1, 2023. Each of the above applications are incorporated by reference in their entirety.

### FIELD OF THE INVENTION

The field of the invention is immunotherapy technologies.

### BACKGROUND

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Autologous or allogenic T-cells and natural killer cells provide meaningful benefit for otherwise refractory malignancies. Apheresis generates the starting material for T cell or NK cell manufacturing. Because NK cells comprise only a small fraction of lymphocytes (˜1-20%), methods have been developed either to enrich them from large volumes of peripheral blood, such as an apheresis product, or to expand the NK cell population from a smaller number of blood or stem cells. As clinical indications for T cells and NK cells are expanding, researchers are trying to develop new and improved ways of obtaining these cells from peripheral blood mononuclear cells (PBMCs).

Thus, there remains a need in the art for improved methods of isolating and expanding T cells and NK cells for treatment of a cancer or infectious disease in a patient in need thereof.

### SUMMARY

The inventive subject matter provides compositions and methods for generating tumor targeted lymphocytes and/or tumor infiltrating lymphocytes and/or tumor targeted natural killer (NK) cells for use in the treatment of a cancer or an infectious disease.

In an embodiment, the inventive subject matter comprises tumor targeted lymphocytes for use in the treatment of a cancer or infectious disease. The tumor targeted lymphocytes are generated by a method comprising the steps of performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction, wherein the remaining apheresis product comprises a CD3−/CD14− fraction; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; and purifying the expanded T cells.

In an embodiment, the inventive subject matter comprises tumor targeted natural killer (NK) cells for use in the treatment of a cancer or infectious disease. The NK cells are generated by a method comprising the steps of performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from the CD3− CD14− fraction of the apheresis; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; purifying the expanded T cells; isolating from the expanded T cells at least one nucleic acid encoding an α and β chain of a T cell receptor (TCR), and fusing the nucleic acid to the 5′ end of a second nucleic acid encoding a transmembrane domain and an intracellular signaling domain of a chimeric antigen receptor (CAR), wherein the fused nucleic acid encodes a TCR CAR; and transfecting the enriched and expanded NK cells with a nucleic acid encoding the TCR CAR.

In an embodiment, the inventive subject matter comprises a method of expanding tumor infiltrating lymphocytes for use in the treatment of a cancer or infectious disease. The method includes performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from the CD3−/CD14− fraction of the apheresis; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; purifying the expanded T cells; purifying CD3+ TIL from a solid tumor and exposing the TIL to the activated DC, thereby expanding the TIL; and purifying the expanded TIL.

In an embodiment, the inventive subject matter comprises a pharmaceutical composition that includes 1) activated dendritic cells (DC), wherein the DC are differentiated from patient apheresis-derived CD14+ monocytes; 2) an adenovirus encoding an antigenic peptide sequence, wherein the DC are activated upon exposure to the adenovirus; and 3) patient apheresis-derived CD3+ T cells, wherein the CD3+ T cells are exposed to the activated DC, thereby activating and expanding the T cells. It is contemplated that the pharmaceutical composition is used in the treatment of a cancer or infectious disease.

In an embodiment, the inventive subject matter comprises a pharmaceutical composition that includes patient-derived dendritic cells (DC), GM-CSF, and an adenovirus (Ad) encoding an antigenic peptide, for use in the treatment of a cancer or infectious disease, wherein the DC are derived from the patient's apheresis.

In an embodiment, the inventive subject matter includes a pharmaceutical composition comprising 1) patient-derived Natural Killer (NK) cells and/or Natural Killer T (NKT) cells, 2) GM-CSF, and 3) an adenovirus (Ad) encoding an antigenic peptide, for use in the treatment of a cancer or infectious disease, wherein the NK or NKT cells are derived from the patient's apheresis.

Further disclosed herein is a pharmaceutical composition comprising 1) patient-derived T cells, B cells, and/or monocytes, 2) GM-CSF, and 3) an adenovirus (Ad) encoding an antigenic peptide, for use in the treatment of a cancer or infectious disease, wherein the T cells, B cells, and/or monocytes are derived from the patient's apheresis.

Also disclosed herein is a pharmaceutical composition comprising 1) dendritic cells (DC), 2) an irradiated biopsy sample, and 3) T-cells, wherein the DC and T-cells are derived from apheresis of a patient, and the biopsy sample is from a tumor of the same patient, and wherein the pharmaceutical composition is for use in the treatment of a cancer.

In an embodiment, the inventive subject matter includes a pharmaceutical composition comprising dendritic cells (DC) and T-cells, wherein the DC and T-cells are isolated from apheresis of a patient, wherein the DC and/or T-cells are exposed to a biopsy sample from a tumor of the same patient, and wherein the pharmaceutical composition is formulated for administration to the patient.

In an embodiment, the inventive subject matter comprises a pharmaceutical composition that includes dendritic cells (DC) and T-cells, wherein an apheresis sample from a patient is exposed to a biopsy sample from a tumor of the same patient, wherein the DC and T-cells are then isolated from the apheresis, and wherein the pharmaceutical composition is formulated for administration to the patient.

Further disclosed herein is a method of generating tumor targeted lymphocytes for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from CD3−/CD14− fraction of the apheresis; differentiating the CD14+ Monocytes into dendritic cells (DC), and exposing to DC to at least one compound selected from the group consisting of a protein arginine methyltransferase 5 (PRMT5) inhibitor, a DNA methyltransferase (DNMT) inhibitor, and a histone deacetylase (HDAC) inhibitor, whereby the DC MHC-I or MHC-II present at least one re-expressed peptide sequence or a portion thereof, thereby activating the DC; and exposing the purified CD3+ T cells to the activated DC, thereby expanding the T cells;

In yet another embodiment, the inventors have disclosed a method of generating tumor targeted natural killer (NK) cell for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from CD3−/CD14− fraction of the apheresis; differentiating the CD14+ Monocytes into dendritic cells (DC), and exposing to DC to at least one compound selected from the group consisting of a protein arginine methyltransferase 5 (PRMT5) inhibitor, a DNA methyltransferase (DNMT) inhibitor, and a histone deacetylase (HDAC) inhibitor, whereby the DC MHC-I or MHC-II present at least one re-expressed peptide sequence or a portion thereof, thereby activating the DC; exposing the purified CD3+ T cells to the activated DC, thereby expanding the T cells; isolating from the expanded T cells at least one nucleic acid encoding an α and β chain of a T cell receptor (TCR), and fusing the nucleic acid to the 5′ end of a second nucleic acid encoding a transmembrane domain and an intracellular signaling domain of a chimeric antigen receptor (CAR), wherein the fused nucleic acid encodes a TCR CAR; transfecting the enriched and expanded NK cells with a nucleic acid encoding the TCR CAR.

In an embodiment, the inventive subject matter comprises a method of expanding tumor infiltrating lymphocytes for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from the CD3−/CD14− fraction of the apheresis; differentiating the CD14+ Monocytes into dendritic cells (DC), and exposing to DC to at least one compound selected from the group consisting of a protein arginine methyltransferase 5 (PRMT5) inhibitor, a DNA methyltransferase (DNMT) inhibitor, and a histone deacetylase (HDAC) inhibitor, whereby the DC MHC-I or MHC-II present at least one re-expressed peptide sequence or a portion thereof, thereby activating the DC; purifying CD3⁺ TIL from a solid tumor and exposing the CD3+ TIL to the activated DC, thereby expanding the TIL; and purifying the expanded TIL.

Also disclosed herein is a pharmaceutical composition comprising tumor targeted CD3⁺ T lymphocytes is disclosed herein, wherein the pharmaceutical composition is for use in the treatment of a cancer or infectious disease. The composition includes 1) dendritic cells (DC), wherein the DC are differentiated from patient-derived apheresis purified CD14+ monocytes, 2) at least one compound selected from the group consisting of a protein arginine methyltransferase 5 (PRMT5) inhibitor, a DNA methyltransferase (DNMT) inhibitor and a histone deacetylase (HDAC) inhibitor, whereby the DC MHC-I or MHC-II present at least one re-expressed peptide sequence or a portion thereof, thereby activating the DC, and 3) CD3⁺ T cells purified from the patient apheresis; wherein the T cells are exposed to the activated DC, thereby activating and expanding the T cells.

Further disclosed herein is a method of expanding tumor infiltrating lymphocytes for use in the treatment of a cancer or infectious disease. The method comprises performing therapeutic apheresis on a subject with cancer or infectious disease, wherein the subject has been treated with at least one therapeutic agent selected from the group consisting of a protein arginine methyltransferase 5 (PRMT5) inhibitor, a DNA methyltransferase (DNMT) inhibitor, and a histone deacetylase (HDAC) inhibitor; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; purifying CD3⁺ TIL from a solid tumor and exposing the TIL to the activated DC, thereby expanding the TIL; purifying the expanded TIL.

In an embodiment, the inventive subject matter comprises a method of generating tumor targeted lymphocytes for use in the treatment of a cancer or infectious disease. The method comprises performing therapeutic apheresis on a subject with cancer or infectious disease, wherein the subject has been treated with at least one therapeutic agent selected from the group consisting of a protein arginine methyltransferase 5 (PRMT5) inhibitor, a DNA methyltransferase (DNMT) inhibitor and a histone deacetylase (HDAC) inhibitor; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; purifying the expanded T cells.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates IncuCyte Image for donor 001 of 12-well Plate of Day 5 Post-transduction.

FIG. 2 illustrates results for donor 001 and shows that different cytokine cocktails (N-803, GM-CSF & IL-4) induce different cell morphologies.

FIG. 3 illustrates results for donor 001 and shows that different cytokine cocktails (N-803, GM-CSF & IL-4) result in different cell morphologies.

FIG. 4 illustrates results for donor 001 and shows that higher number of GFP+ cells were observed in GM-CSF Treatment Group on Day 3 & 5 Post-transduction.

FIG. 5 illustrates IncuCyte Image for donor 002 of 12-well Plate of Day 5 Post-transduction.

FIG. 6 illustrates results for donor 002 and shows that different cytokine cocktails (N-803, GM-CSF & IL-4) induce different cell morphologies.

FIG. 7 illustrates results for donor 002 and shows that higher number of GFP+ cells were observed in GM-CSF Treatment Group on Day 3 & 5 Post-transduction.

FIG. 8 illustrates the flow cytometry gating strategy.

FIG. 9 shows a treatment chart for locally advanced neoadjuvant pancreatic cancer.

### DETAILED DESCRIPTION

The inventive subject matter provides compositions and methods by which dendritic cells from a patient (cancer patient or patient having viral infection) are exposed to pharmacologic manipulation of epigenetic modulators. In particular, protein arginine methyltransferase 5 (PRMT5), DNA methyltransferase (DNMT), and histone deacetylase (HDAC) mediate epigenetic events. These proteins have been shown, in vitro and in vivo (pre-clinical and clinical), to modulate activity of genes involved with both tumor cell proliferation and tumor suppression. Thus, epigenetic modulation has become a clinically validated target for reducing tumor cell proliferation and inducing tumor suppression. Just as genes for tumor suppression can be epigenetically silenced, the inventors have conceived and presented herein the epigenetic silencing of immunogenic peptides, whereby mutations which would routinely be surveilled by the cell-mediated immune system are silenced. Treatment with epigenetic inhibitors facilitate re-expression of immunogenic peptides derived from mutant genes or cells infected with an infectious agent.

As further described in this disclosure, apheresis product is processed to purify CD3+ T cells, CD14+ Monocytes and use of CD3− CD14−cells to enrich and expand NK cells. The CD14+ cells are used to differentiate into dendritic cells which are then exposed to at least one epigenetic modulation agent, wherein the agent selected from the group consisting of a protein arginine methyltransferase 5 (PRMT5) inhibitor, a DNA methyltransferase (DNMT) inhibitor and a histone deacetylase (HDAC) inhibitor. The presence of the agent induces expression of neoepitopes which are presented by MHC-I and MHC-II at the cell surface. Subsequent exposure to the T cells results in activation and expansion of T cells which are specific for the newly expressed neoepitopes. The same logic applies regarding expression of infectious agent epitopes.

It is also contemplated that the patient is treated with at least one epigenetic modulator selected from the group consisting of a protein arginine methyltransferase 5 (PRMT5) inhibitor, a DNA methyltransferase (DNMT) inhibitor and a histone deacetylase (HDAC) inhibitor. Apheresis product is derived from the treated patient and separated into dendritic cells and T lymphocytes. Dendritic cells are re-expressing suppressed neoepitopes or immunogenic peptides for stimulation of the isolated T cells. Epigenetic modulation agents thereby unmask genes which encode tumor promoting peptides (cell cycle inducers) and/or tumor specific peptides (neoepitopes), both of which stimulate, via MHC presentation, the activation and proliferation of tumor educated lymphocytes.

In another embodiment, NK cells are isolated from the apheresis product, activated and expanded ex vivo, and reinfused into the patient along with tumor or virally educated T cells.

In another embodiment, the purified dendritic cells are treated with IL-15 or an agonist derivative thereof, such as N-803. The dendritic cells may be from a patient who has been treated with epigenetic modulators, or the dendritic cells may be treated with epigenetic modulators ex vivo. The dendritic cells may be from a patient who has been treated with epigenetic modifiers and/or an IL15 agonist derivative, particularly a stabilized derivative thereof.

In another embodiment, the patient may receive along with T cell and/or NK cell therapies IL-15 or a stabilized agonist derivative thereof. Tumor or virally educated lymphocyte administration may be supplemented with chemotherapeutics, tumor targeted antibodies, checkpoint inhibitor antibodies, vaccines (adenoviral or yeast-based), or radiation.

In one aspect, the present disclosure provides methods of generating tumor targeted lymphocytes for use in the treatment of a cancer or infectious disease. The method comprises performing therapeutic apheresis on a subject with cancer or infectious disease. In this regard, it should be recognized that apheresis is conducted by obtaining circulating blood from a person, passing the blood through an apparatus that separates the red and white blood cells (apheresis product) from the plasma, and returning the plasma back to the patient's circulation. The apheresis product is then separated into two fractions: a purified CD3+ T cell fraction, and a remaining apheresis product fraction that comprises CD3−cells. The CD3− fraction is then further separated into two fractions: a purified CD14+ monocytic cells fraction, and a remaining apheresis product fraction that comprises CD3− CD14−cells. The purified CD14+ monocyte cell fraction is differentiated into dendritic cells (DC). The DCs are then exposed to or transfected with one or more antigenic peptides that are related to the patient's cancer or infectious disease, or with an expression vector (preferably viral vector) that includes a nucleic acid that encodes the one or more antigenic peptides. Most preferably, and as further discussed in more detail below, the tumor-related neoepitopes include or are neoepitopes specific to the patient's tumor, while virus-related neoepitopes are specific to the virus and the patient. As a result, the so exposed or transfected dendritic cells will present the tumor epitopes via the MHC-I/MHC-II system, thereby activating the DC. The previously purified CD3+ T cell fraction is then exposed to the activated DC, to thereby expand the T cells. Finally, the expanded T cells are purified for use in the treatment of the cancer or infectious disease.

Optionally, once the CD14+ cells are differentiated into dendritic cells, they are exposed to at least one epigenetic modulation agent, wherein the agent selected from the group consisting of a protein arginine methyltransferase 5 (PRMT5) inhibitor, a DNA methyltransferase (DNMT) inhibitor and a histone deacetylase (HDAC) inhibitor. The presence of the at least one epigenetic modulation agent induces expression of tumor-and patient-specific neoepitopes or virus specific neoepitopes, which are presented by MHC-I and MHC-II at the cell surface. Subsequent exposure to the T cells results in activation and expansion of T cells which are specific for the newly expressed tumor-and patient-specific neoepitopes or virus specific neoepitopes.

Optionally, prior to performing therapeutic apheresis on the subject, the subject may be treated with at least one therapeutic agent selected from the group consisting of a protein arginine methyltransferase 5 (PRMT5) inhibitor, a DNA methyltransferase (DNMT) inhibitor and a histone deacetylase (HDAC) inhibitor.

The above disclosed method may further be used for generating tumor targeted natural killer (NK) cell for use in the treatment of a cancer or infectious disease. In this embodiment, NK cells are expanded from the CD3− CD14− fraction of the apheresis. Then, from the expanded T cells, a nucleic acid encoding an α and β chain of a T cell receptor (TCR) is isolated. This nucleic acid is then fused to the 5′ end of a second nucleic acid encoding a transmembrane domain and an intracellular signaling domain of a chimeric antigen receptor (CAR). In this way, a fused nucleic acid that encodes a TCR CAR is obtained. The enriched and/or expanded NK cells are then transfected with the fused nucleic acid encoding the TCR CAR to thereby generate a tumor or infectious disease targeted natural killer (NK) cell for use in the treatment of the cancer or infectious disease.

In some embodiments, the above disclosed method may be used for expanding tumor infiltrating lymphocytes (TIL) for use in the treatment of a cancer or infectious disease. In this case, CD3⁺ TIL are obtained from a solid tumor, and the CD3+ TIL are exposed to the activated DC, thereby expanding the TIL. The expanded TIL is then purified and used for treatment of a cancer or infectious disease.

In another aspect, the present disclosure provides a pharmaceutical composition comprising one or more of the following components: 1) activated dendritic cells (DC), wherein the DC are differentiated from patient apheresis-derived CD14+ monocytes; 2) an adenovirus encoding an antigenic peptide sequence; 3) patient apheresis-derived CD3+ T cells, 4) an irradiated biopsy sample. 5) at least one compound selected from the group consisting of a protein arginine methyltransferase 5 (PRMT5) inhibitor, a DNA methyltransferase (DNMT) inhibitor and a histone deacetylase (HDAC) inhibitor; 6) granulocyte-macrophage colony-stimulating factor (GM-CSF); 7) patient-derived Natural Killer (NK) cells, Natural Killer T (NKT) cells, T cells, B cells, and/or monocytes. In preferred embodiments, the pharmaceutical composition comprises at least two, or at least three, or at least four, or at least five of the above components.

In a preferred embodiment, the pharmaceutical composition comprises tumor or viral disease targeted lymphocytes for use in the treatment of a cancer or infectious disease. The composition comprises 1) activated dendritic cells (DC), wherein the DC are differentiated from patient apheresis-derived CD14+ monocytes; 2) an adenovirus encoding an antigenic peptide sequence, wherein the DC are activated upon exposure to the adenovirus; and 3) patient apheresis-derived CD3+ T cells, wherein the CD3+ T cells are exposed to the activated DC, thereby activating and expanding the T cells. Alternatively or additionally, the pharmaceutical composition may comprise patient-derived dendritic cells (DC), GM-CSF, and an adenovirus (Ad) encoding an antigenic peptide, for use in the treatment of a cancer or infectious disease, wherein the DC are derived from the patient's apheresis.

In yet another embodiment, the pharmaceutical composition comprises 1) patient-derived Natural Killer (NK) cells and/or Natural Killer T (NKT) cells, 2) GM-CSF, and 3) an adenovirus (Ad) encoding an antigenic peptide, for use in the treatment of a cancer or infectious disease, wherein the NK or NKT cells are derived from the patient's apheresis. Alternatively or additionally, the pharmaceutical composition may comprise 1) patient-derived T cells, B cells, and/or monocytes, 2) GM-CSF, and 3) an adenovirus (Ad) encoding an antigenic peptide, for use in the treatment of a cancer or infectious disease, wherein the T cells, B cells, and/or monocytes are derived from the patient's apheresis.

It is further contemplated that the pharmaceutical composition may comprise 1) dendritic cells (DC), 2) an irradiated biopsy sample, and 3) T-cells, wherein the DC and T-cells are derived from apheresis of a patient, and the biopsy sample is from a tumor of the same patient, and wherein the pharmaceutical composition is for use in the treatment of a cancer. Alternatively or additionally, the pharmaceutical composition may comprise dendritic cells (DC) and T-cells, wherein the DC and T-cells are isolated from apheresis of a patient, wherein the DC and/or T-cells are exposed to a biopsy sample from a tumor of the same patient, and wherein the pharmaceutical composition is formulated for administration to the patient.

It is also contemplated that the pharmaceutical composition may comprise dendritic cells (DC) and T-cells, wherein an apheresis sample from a patient is exposed to a biopsy sample from a tumor of the same patient, wherein the DC and T-cells are then isolated from the apheresis, and wherein the pharmaceutical composition is formulated for administration to the patient. Alternatively or additionally, the pharmaceutical composition may comprise tumor targeted CD3⁺ T lymphocytes for use in the treatment of a cancer or infectious disease, the composition comprising 1) dendritic cells (DC), wherein the DC are differentiated from patient-derived apheresis purified CD14+ monocytes, 2) at least one compound selected from the group consisting of a protein arginine methyltransferase 5 (PRMT5) inhibitor, a DNA methyltransferase (DNMT) inhibitor and a histone deacetylase (HDAC) inhibitor, whereby the DC MHC-I or MHC-II present at least one re-expressed peptide sequence or a portion thereof, thereby activating the DC, and 3) CD3+ T cells purified from the patient apheresis; wherein the T cells are exposed to the activated DC, thereby activating and expanding the T cells.

In the present disclosure, the CD3+ T cells are obtained (and purified) from a tumor or from a blood sample of the patient and expanded ex-vivo. The thusly expanded T cells may be reintroduced to the patient as autologous cells or may be administered to a different subject as donor cells or allogenic cells.

The T cells may be expanded by exposure to a cytokine cocktail comprising one or more of IL-2, IL-15, and IL-7, or an agonist derivative thereof. Alternatively, the T cells may genetically modified to express endoplasmic reticulum localized IL-15 (erIL-15). In this case, the genetic modification of the T cells comprises introducing into the T cell a nucleic acid that encodes a cytokine such as IL-2 or IL-15. The IL-2 may be expressed with a signal sequence that directs the IL-2 to the endoplasmic reticulum IL-2 (“erIL-2”). Similarly, the IL-15 may be expressed with a signal sequence that directs the IL-15 to the endoplasmic reticulum IL-15 (“erIL-15”). This permits expression of IL-2 and/or IL-15 at levels sufficient for autocrine activation, but without releasing IL-2 extracellularly. See Konstantinidis et al “Targeting IL-2 to the endoplasmic reticulum confines autocrine growth stimulation to NK-92 cells” Exp Hematol. 2005 February; 33(2):159-64

Furthermore, in some embodiments, T cells are genetically modified to express a chimeric antigen receptor (CAR), and wherein the CAR targets a tumor antigen or a checkpoint inhibitor. The intracellular signaling domain of the CAR may also comprise an FcεRIγ portion. U.S. patent application Ser. No. 17/341,098, which discloses such methods, is herein incorporated by reference. The T cell is preferably engineered to express a TCR which recognizes MHC-I presented peptides. Preferably, the T cells are genetically modified by transfecting the T cells with one or more nucleic acids that encodes the one or more CARs. Transfection techniques include, but are not limited to viral transduction, mRNA transfection, and the Sleeping Beauty transposon system. Subsequent to transfection, CAR T cells may be expanded in a bioreactor until a clinically effective number of cells is obtained.

The expression vector may be a viral vector, and preferably an adenovirus such as an Ad5 adenovirus. Moreover, it is further generally preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 February; 72(2): 926-933). Most typically, the desired nucleic acid sequences (for expression from virus infected cells) are under the control of appropriate regulatory elements well known in the art. The patent application PCT/US2017/045093 discloses such methods.

Furthermore, the dendritic cells may be further exposed to one or more of a modified RNA, a lentivirus, and/or to a peptide pool of neoepitopes.

The term “epigenetics” describe heritable changes in a cellular phenotype without changes in genotype. Epigenetic modifications generally refer to the alterations in gene expression without altering the DNA sequence. These modifications include DNA and RNA methylation, histone modifications, chromatin remodeling, and noncoding RNA.

Epigenetic regulation is a dynamic and reversible process characterized by the addition and removal of modifications to DNA and histones. In most cases, the modifications are covalent modifications to DNA and histones. These modifications are carried out by chromatin-modifying enzymes in a tightly regulated and cooperative manner, resulting in changes to the structure of chromatin. The regulators responsible for these epigenetic modifications on DNA and histones have been classified into four main categories: “writers,” “erasers,” “readers,” or “movers.” Writers introduce epigenetic marks onto DNA or histones, including DNA methyltransferases (DNMTs), histone methyltransferases (HMTs), and histone acetyltransferases (HATs). Erasers, on the other hand, remove epigenetic marks through the action of histone lysine demethylases (KDMs) and histone deacetylases (HDACs). Readers recognize or are recruited to specific epigenetic marks, such as chromodomain and bromodomain (BRD) proteins recognizing methylated or acetylated residues, respectively. Movers are chromatin-remodeling proteins that change the dynamic spatiotemporal positioning of nucleosomes to allow for gene transcription.

As discussed by the inventors herein, dysregulation of epigenetic modifications can lead to the activation of oncogenes or the silencing of tumor suppressor genes, as well as disruption of multiple signaling pathways.

DNMTs are a type of writing enzymes that play a role in DNA methylation. Writing enzymes like DNMT1, DNMT3a, and DNMT3b add methyl groups to cytosine residues in DNA. Despite having a similar structure with a regulatory domain at the N-terminal and a catalytic domain at the C-terminal, these enzymes differ in their functions and expression patterns. As a maintenance methyltransferase, DNMT1 cannot only maintain the stability of already methylated DNA sequences, ensuring their preservation during DNA replication and cell division, but also repair DNA methylation. In contrast, DNMT3a and DNMT3b are known as de novo methyltransferases, which can add new methyl groups to previously unmethylated DNA sequences, thereby forming new methylation patterns. DNMT3a/b are targeted to specific DNA sequences by TFs like CTCF, Sp1, YY1, NRSF/REST, FOXA1, and SALL4.11 Two other DNMTs, DNMT2 and DNMT3L, do not have cytosine methyltransferase activity. DNMT3L can increase the binding capacity of DNMT3a and DNMT3b to the methyl donor S-adenosyl-1-methionine (SAM) to enhance their activity. DNMT2 primarily functions by introducing methyl chains into ncRNAs such as transfer RNA, ribosomal RNA, and nuclear RNA.

PRMTs are a type of histone methylation family of enzymes. RMTs have been implicated in a broad spectrum of disease models, including neurological disorders, inflammatory diseases, cardiovascular diseases, and cancer. In particular, aberrant expression of PRMTs has been extensively studied in cancer, such as lung cancer, breast cancer, CRC, and leukemia. PRMTs are frequently overexpressed in various tumor types, including breast cancer and prostate cancer, and has been shown to promote tumor growth and metastasis. PMRT dysregulation is believed to play a critical role in cancer initiation and progression, highlighting its potential as a therapeutic target for cancer treatment.

HDACs possess the ability to eliminate acetyl groups from lysine residues on both histone and nonhistone proteins, resulting in a more compact chromatin structure and reduced transcription activity. The overexpression of HDACs is frequently observed in many cancer patients. In addition to alterations in expression or genetic, HDACs can be abnormally recruited to specific gene promoters by oncogenic fusion proteins to drive leukemogenesis. Moreover, high expression of HDACs has been associated with drug resistance in various cancers. For example, HDAC increases temozolomide resistance in glioblastoma, as well as cisplatin and sorafenib resistance in NSCLC. Similarly, HDAC, which are significantly upregulated in glioblastoma, contribute to resistance to temozolomide chemotherapy. Taken together, abnormal expression of HDACs contributes to tumor resistance development, and inhibiting these enzymes may prevent the emergence of drug resistance.

Epigenetic drugs currently approved by the FDA for cancer include Azacitidine (5-azacytidine) Decitabine (5-Aza-2′-deoxycytidine), Vorinostat (Suberoylanilide hydroxamic acid (SAHA)), Romidepsin (Depsipeptide), Belinostat (Beleodaq, PXD101), Panobinostat (LBH589), Chidamide (Tucidinostat), Tazemetostat (EPZ-6438), Enasidenib (AG-221), and Ivosidenib (AG-120), as disclosed in Tao L, Zhou Y, Luo Y, et al. Epigenetic regulation in cancer therapy: from mechanisms to clinical advances. MedComm-Oncology. 2024; 3: e59. doi:10.1002/mog2.59, the entire content of which is incorporated by reference herein.

The term “apheresis” generally refers to removing whole blood from a patient or donor and separating the blood into two or more components. In an apheresis procedure, blood is withdrawn from a subject through a needle inserted into the vein. The needle is attached to one end of a plastic tube which provides a flow path for the blood. The other end of the tube terminates in a container for collecting the blood. The collected blood is then separated in a separator, such as a centrifuge, into its components. The desired blood component which, depending on the procedure, can be red blood cells, platelets, plasma, white blood cells or stem cells, may be collected. These components are further processed to purify blood fractions such as the CD3+ fraction, the CD3− fraction, the CD3− CD14− fraction, CD14+ fraction etc. One or more of these different fractions are further modified/processed as discussed throughout this application. One or more of these fractions may be transfused back to a patient in need of the component/fraction.

With respect to NK cells, it should be noted that all NK cells are deemed suitable for use herein and therefore include primary NK cells (preserved, expanded, and/or fresh cells), secondary NK cells that have been immortalized, autologous or heterologous NK cells (banked, preserved, fresh, etc.), and modified NK cells as described in more detail below. In some embodiments, it is preferred that the NK cells are NK-92 cells. The NK-92 cell line is a unique cell line that was discovered to proliferate in the presence of interleukin 2 (IL-2) (see e.g., Gong et al., Leukemia 8:652-658 (1994)). NK-92 cells are cancerous NK cells with broad anti-tumor cytotoxicity and predictable yield after expansion in suitable culture media. Advantageously, NK-92 cells have high cytolytic activity against a variety of cancers.

The original NK-92 cell line expressed the CD56bright, CD2, CD7, CD11a, CD28, CD45, and CD54 surface markers and did not display the CDI, CD3, CD4, CD5, CD8, CD10, CD14, CD16, CD19, CD20, CD23, and CD34 markers. Growth of such NK-92 cells in culture is dependent upon the presence of interleukin 2 (e.g., rIL-2), with a dose as low as 1 IU/mL being sufficient to maintain proliferation. IL-7 and IL-12 do not support long-term growth, nor have various other cytokines tested, including IL-la, IL-6, tumor necrosis factor α, interferon α, and interferon γ. Compared to primary NK cells, NK-92 typically have a high cytotoxicity even at relatively low effector:target (E:T) ratios, e.g. 1:1. Representative NK-92 cells are deposited with the American Type Culture Collection (ATCC), designation CRL-2407. U.S. Pat. No. 7,618,817, U.S. Pat. No. 8,034,332, U.S. Pat. No. 8,313,943, U.S. Pat. No. 9,150,636, U.S. Pat. No. 9,181,322, U.S. Pat. No. 10,138,462, and U.S. Pat. No. 10,258,649 are herein incorporated by reference in their entirety as are all other extrinsic references.

In another aspect of the inventive subject matter, the genetically engineered NK cell may also be an NK-92 derivative that is modified to express the high-affinity Fcγ receptor (CD16). Sequences for high-affinity variants of the Fcγ receptor are well known in the art (see e.g., Blood 2009 113:3716-3725), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells. Such cells may then be additionally genetically modified to a CAR as further described in more detail below. U.S. Pat. No. 10,738,279, U.S. Pat. No. 10,456,420, U.S. Pat. No. 10,736,921, U.S. Pat. No. 11,000,550, U.S. Pat. No. 10,801,013, and U.S. Pat. No. 10,774,310

Genetic modification of the NK cells contemplated herein can be performed in numerous manners, and all known manners are deemed suitable for use hereon. Moreover, it should be recognized that NK cells can be transfected with DNA or RNA, and the particular choice of transfection will at least in part depend on the type of desired recombinant cell and transfection efficiency. For example, where it is desired that NK cells are stably transfected, linearized DNA may be introduced into the cells for integration into the genome. On the other hand, where transient transfection is desired, circular DNA or linear RNA (e.g., mRNA with polyA+ tail) may be used.

For example, where the NK cell is an autologous NK cell or an NK-92 cell it is contemplated that the recombinant nucleic acid will include a segment that encodes a CAR that includes FcεRIγ signaling domain, and preferably also a segment that encodes a cytokine to provide autocrine growth stimulation (e.g., IL-2, IL-2 that is modified with an ER retention sequence, IL-15, or IL-15 that is modified with an ER retention sequence) and/or a segment that encodes a CD16 or high affinity CD16158V. As will be readily appreciated, inclusion of a cytokine that provides autocrine growth stimulation will render the modified recombinant independent of exogenous cytokine addition, which will render large scale production of such cells economically feasible. Likewise, where the modified recombinant also expresses CD16 or a high affinity CD16158V, such cells will have further enhanced ADCC characteristics and with that further improved targeted cytotoxicity.

One should appreciate that the recombinant nucleic acid that encodes that cytokine and/or the CD16 or high affinity CD16158V can be integrated in to the genome of the NK cell, or can be supplied as an extrachromosomal unit (which may be a linear or circular DNA, or a linear RNA, virally delivered or via chemical, mechanical, or electrical transfection). For example, recombinant NK-92 cells expressing IL-2ER and CD16158V are known as haNK cells (Oncotarget 2016 Dec. 27; 7(52): 86359-86373) and can be transfected with a recombinant nucleic acid that includes a segment that encodes a CAR that includes FcεRIγ signaling domain. Once more, such recombinant nucleic acid may comprise further segments that may encode additional immunotherapeutic proteins, such as N-803, TxM-type compounds, IL-8 traps, TGF-β traps, etc. Likewise, NK-92 cells may already be transfected with a cDNA that encodes IL-2 (e.g., NK-92 MI, ATCC CRL-2408). Such cells can then be further transfected with a recombinant nucleic acid that includes a segment that encodes a CAR that includes FcεRIγ signaling domain along with a segment that encodes a CD16 or high affinity CD16158V.

On the other hand, (e.g., autologous, fresh, cultivated, or previously frozen) NK cells or NK-92 cells may also be transfected with a recombinant nucleic acid that includes a segment that encodes a CAR with a FcεRIγ signaling domain, a segment that encodes a cytokine to provide autocrine growth stimulation (e.g., IL-2, IL-2 that is modified with an ER retention sequence, IL-15, or IL-15 that is modified with an ER retention sequence) and a segment that encodes a CD16 (SEQ ID NO:34) or high affinity CD16158V (SEQ ID NO:35, encoded by SEQ ID NO:36) as further disclosed in PCT/US2019/033407, which is incorporated by reference herein in its entirety. Most typically, such recombinant nucleic acid will be arranged as a tricistronic construct. As noted before, such constructed can be an extrachromosomal circular plasmid, a linear DNA (which may be integrated into the genome of the NK cell), or a linear RNA. Such nucleic acids will typically be transfected into the cells in a manner well known in the art (e.g., electroporation, lipofection, ballistic gene transfer, etc.). Similarly, the nucleic acid may be delivered to the cell via a recombinant virus. Therefore, NK cells suitable for use herein include NK-92 cells (which may be transfected with a tricistronic construct encoding a CAR, a CD16 or variant thereof, and a cytokine or variant thereof), a genetically modified NK cell or NK-92 cell that expresses a CD16 or variant thereof or a cytokine or variant thereof (which may be transfected with a nucleic acid encoding a CAR and a CD16 or variant thereof or a cytokine or variant thereof), and a genetically modified NK cell or NK-92 cell that expresses a CD16 or variant thereof and a cytokine or variant thereof (which may be transfected with a nucleic acid encoding a CAR). U.S. patent application Ser. No. 17/056,385 and U.S. Pat. No. 11,077,143, U.S. Pat. No. 10,738,279, U.S. patent application Ser. 16/969,152 are herein incorporated by reference.

In preferred embodiments, it should therefore be noted that the genetically modified NK cell (especially where the cell expresses a CAR and CD16 or variant thereof) will exhibit three distinct modes of cell killing: General cytotoxicity which is mediated by activating receptors (e.g., an NKG2D receptor), ADCC which is mediated by antibodies bound to a target cell, and CAR mediated cytotoxicity.

Consequently, it should be appreciated that the manner of transfection will at least in part depend on the type of nucleic acid employed. Therefore, viral transfection, chemical transfection, mechanical transfection methods are all deemed suitable for use herein. For example, in one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In another embodiment, the vectors described herein allow for stable transfection of cells. In one embodiment, the vector allows incorporation of the transgene(s) into the genome of the cell. Preferably, such vectors have a positive selection marker and suitable positive selection markers include any genes that allow the cell to grow under conditions that would kill a cell not expressing the gene. Non-limiting examples include antibiotic resistance, e.g. geneticin (Neo gene from Tn5). Alternatively, or additionally, the vector is a plasmid vector. In one embodiment, the vector is a viral vector, and preferably an adenoviral vector. As would be understood by one of skill in the art, any suitable vector can be used, and suitable vectors are well-known in the art.

In still other embodiments, the cells are transfected with mRNA encoding the protein of interest (e.g., the CAR). Transfection of mRNA results in transient expression of the protein. In one embodiment, transfection of mRNA into NK-92 cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration. In at least some embodiments as described in more detail below, NK cell transfection with mRNA resulted in unexpectedly consistent and strong expression of the CAR at a high faction of transfected cells. Moreover, such transfected cells also exhibited a high specific cytotoxicity at comparably low effector to target cell ratios.

With respect to contemplated CARs it is noted that the NK or NK-92 cells will be genetically modified to express the CAR as a membrane bound protein exposing a portion of the CAR on the cell surface while maintaining the signaling domain in the intracellular space. Most typically, the CAR will include at least the following elements (in order): an extracellular binding domain, a hinge domain, a transmembrane domain, and an FcεRIγ signaling domain.

In preferred embodiments, the cytoplasmic domain of the CAR comprises or consists of a signaling domain of FcεRIγ. Notably, and as described in more detail below, the FcεRIγ signaling domain provide for substantially increased expression levels of the CAR as much as for significantly extended cytotoxicity over time. In some embodiments, the FcεRIγ cytoplasmic domain is the sole signaling domain. However, it should be appreciated that additional elements may also be included, such as other signaling domains (e.g., CD28 signaling domain, CD3ζ signaling domain, 4-1BB signaling domain, etc.). These additional signaling domains may be positioned downstream of the FcεRIγ cytoplasmic domain and/or upstream of the FcεRIγ cytoplasmic domain. In alternative embodiments, the cytoplasmic domain of the CAR may also comprise a signaling domain of CD3 zeta (CD3ζ). In one embodiment, the cytoplasmic domain of the CAR consists of a signaling domain of CD3 zeta.

Therefore, contemplated CARs will include a general structure of a desired antigen binding domain that is coupled to a hinge domain, which is coupled to a transmembrane domain, which is coupled to a signaling domain. Viewed from another perspective, contemplated CARs may have a desired binding domain that is then coupled to a hybrid protein that comprises, consists of, or essentially consists of a hinge domain, which is coupled to a transmembrane domain, which is coupled to a signaling domain.

Most typically, but not necessarily, the extracellular binding domain of the CAR will be a scFv or other natural or synthetic binding portion that specifically binds an antigen of interest. Especially suitable binding portions include small antibody fragments with single, dual, or multiple target specificities, beta barrel domain binders, phage display fusion proteins, etc. Among other suitable extracellular binding domains, preferred domains will specifically bind to a tumor-specific antigen, a tumor associated antigen, or a patient-and tumor-specific antigen. Tumor-specific antigens include, without limitation, NKG2D ligands, CS1, GD2, CD138, EpCAM, EBNA3C, GPA7, CD244, CA-125, ETA, MAGE, CAGE, BAGE, HAGE, LAGE, PAGE, NY-SEO-1, GAGE, CEA, CD52, CD30, MUC5AC, c-Met, EGFR, FAP, WT-1, PSMA, NY-ESO1, AFP, CEA, CTAGIB, and CD33. Additional non-limiting tumor-associated antigens, and the malignancies associated therewith, can be found in Table 1. Still further tumor-specific antigens are described, by way of non-limiting example, in US2013/0189268; WO 1999024566 A1; U.S. Pat. No. 7,098,008; and WO 2000020460, each of which is incorporated herein by reference in its entirety. Likewise, other preferred domains will specifically bind to a (pathogenic) virus-specific antigen, such as an antigen of an HIV virus (e.g., gp120), an HPV virus, an RSV virus, an influenza virus, an ebolavirus, or an HCV virus.

Consequently, contemplated CARs will target antigens associated with a specific cancer type. For example, targeted cancers include leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodendroglioma, menangioma, melanoma, neuroblastoma, and retinoblastoma. Examples of infectious diseases that are contemplated to be treatment with the present methods include AIDS, H1N1, Ebola, BSE, Zika, SARS, coronavirus. hepatitis A, hepatitis B, hepatitis C, hepatitis D, and hepatitis E.

In an embodiment, the inventive subject matter comprises a method of treatment for cancer, the method comprising determining the MHC-I expression levels in a tumor. Upon determination that MHC-I levels are low relative to control non-cancerous tissue, the patient is administered a plurality of NK cells, wherein the NK cells comprise at least one of an aNK cell, a haNK cell, a t-haNK cell, or a primary ceNK or a memory-like ceNK (m-ceNK) cell. NK cells may be autologous or allogeneic. The NK cell may be administered intravenously or intratumorally. After a period of time, the patient is administered a plurality of T cells. The T cells may be autologous or allogeneic. The T cell may further be genetically engineered to express a targeting agent, wherein the agent comprises a CAR or a TCR.

A suitable method for determining MHC-I levels in a tumor and normal tissue sample involves measuring cell surface expression of MHC-I. Enzymatic or mechanical dissociation of tissues, whereby viable whole cells are separated and purified from primary tissue, is required for Ab staining and flow cytometric determination of MHC-I surface expression. Alternative methods may include transcriptomic analysis, proteomics, western blotting, and surface plasmon resonance (SPR).

As used herein, a t-haNK cell is an NK cell expressing a genetically engineered CAR. Without being bound by any particular theory, it is understood that the targeting moiety on the CAR is dual purpose. First, the targeting moiety directs the NK cell to the site of expression of the antigen in the patient, thereby facilitating delivery of the NK cell to the tumor tissue. Second, the targeting moiety may be immunogenic, thereby facilitating delivery of antibodies and T cells to the tumor.

Primary NK cells may also be enriched and expanded from whole or cord blood mononuclear cells via standard methods including exposure of the primary NK cells to a CD16 antibody, dexamethasone, and/or IL-15. Stabilized IL-15 may be used, wherein stabilized IL-15 includes IL-15 superagonists such as nogapendenkin alpha imbakicept (Alt-803, N-803, Vesanktiva), as well as stabilized IL-15/IL-15Ra fusion proteins. U.S. patent application Ser. No. 16/985,728, U.S. patent application Ser. No. 16/505,528, U.S. Pat. No. 11,351,196, U.S. patent application Ser. No. 63/156,269 U.S. Pat. No. 8,163,879, U.S. Pat. No. 8,507,222, and U.S. Pat. No. 10,537,615 are herein incorporated by reference.

The cytokine enhanced NK (ceNK) cells disclosed herein refers to NK cells in which the cytotoxic activity is enhanced by cytokine stimulation. ceNK cells are prepared by inducing NK cells with a corticosteroid and optionally a cytokine composition comprising IL-15, IL-15:IL-15Ra, or agonist derivatives thereof, such as N-803. The cytokine composition may comprise a fusion protein, wherein the fusion protein comprises IL-15 or an agonist derivatives thereof. Fusion proteins comprising IL-15, wherein the fusion protein has increased stability over IL-15 are preferred. While not limiting the inventive subject matter, it is generally preferred that the corticosteroid is hydroxycortisone and the optional cytokine is N-803.

The Memory-like Cytokine-Enhanced NK Cells (m-ceNK) disclosed herein comprises enriched and expanded NK cells obtained from peripheral blood of donors using the apheresis technique to generate NK cells with a memory-like phenotype. The m-ceNK cells exhibit both high cytotoxicity and increased interferon-gamma production. These m-ceNK cells can be generated from an individual donor for autologous cell therapy or may be generated as an allogeneic product from cord blood. In addition to the enhanced efficacy, m-ceNK cells can be infused easily in an outpatient setting.

As a non-limiting example, the m-ceNK cells may be generated by a step of obtaining a plurality of mononuclear cells and contacting the plurality of mononuclear cells with a corticosteroid and optionally a cytokine. In another step, the plurality of mononuclear cells are incubated in the presence of the corticosteroid and the optional cytokine to enrich the mononuclear cells in NK cells, and the enriched NK cells are then induced with a cytokine composition comprising IL-15, IL-12, and IL-18, or agonist derivatives thereof. The composition may comprise one or more fusion proteins, wherein the fusion proteins comprise at least one of the IL-15, IL-12, and IL-18 cytokines, or agonist derivatives thereof. The cytokine composition may comprise a TxM fusion protein to generate the m-ceNK cells, wherein the TxM fusion protein comprises a protein portion having IL-12 activity, a protein portion having IL-15 activity, and a protein portion having IL-18 activity.

Further description of making the m-ceNK cells and its advantageous properties is described in PCT/US2022/018290, which is incorporated by reference in its entirety. U.S. patent application Ser. No. 17/375,985 and U.S. Pat. No. 11,453,862 provide additional alternative methods of inducing enrichment and expansion of NK cells. Each of the above references are incorporated by reference in its entirety.

“T cell receptor” or “TCR” refers to a dimeric polypeptide that is typically found on the surface of T cells. Each peptide chain of a TCR generally comprises an extracellular domain comprising a variable region and a constant region, a transmembrane domain, and an intracellular domain. The variable region is the portion of the TCR that interacts with the antigen presented by the MHC. The constant region is the area in each of the two peptides wherein the two peptide chains are covalently linked by a disulfide bond. The intracellular domain generally comprises a CD3z, which comprises one or more immunoreceptor tyrosine-based activation motifs (ITAMs). The ITAM mediates the binding of the variable region to the appropriate intracellular signaling pathways.

The intracellular signaling domain of the CAR may also comprise an FcεRIγ portion. U.S. patent application Ser. No. 17/341,098 is herein incorporated by reference.

A T cell may optionally comprise a modified TCR, which relates to a dimeric polypeptide based on a TCR structure. In particular, the modified TCR comprises two peptide chains, each of which comprise an extracellular domain (comprising a variable region, a constant region, and a connecting peptide), a transmembrane domain, and an intracellular domain. In a specific embodiment, the variable region and constant region are attached via a linker. In another specific embodiment, the connecting peptide is located between the constant region and the transmembrane domain. In a further specific embodiment, the two peptide chains are connected to each other by a disulfide bond between the connecting peptides of each peptide chain. The modified TCR does not interact with endogenous TCR's produced by the T cell. The contents of U.S. Patent Application No. 63/227,195 is herein incorporated by reference.

Once the CAR-based therapeutic (e.g., an antigen binding domain coupled to a CAR scaffold) binds to the antigen expressed by the cancer cell, the cytotoxic cell can trigger destruction of the cancer cell. While it is generally contemplated that all cytotoxic cells are deemed suitable for use herein, especially preferred cytotoxic cells include NK cells, activated NK cells, high affinity NK cells, CD8+ T-cells, and CD4+ T-cells that have been modified to recombinantly express the CAR-based therapeutic, any of which may be of different origins. The cytoxic cell is engineered to express a TCR which recognizes MHC-I presented peptides.

Therapeutic T cells as used herein may be patient-derived (autologous) or donor derived (allogeneic). T cells are typically obtained via leukapheresis, and further separated according to surface marker (CD4, CD8) expression. Purified T cells may be activated by exposure to CD3 and/or CD28 Ab's, or by exposure to antigen presenting cells (APC). Cells may be expanded by exposure to a cytokine cocktail comprising one or more of IL-2, IL-15, and IL-7. In a preferred embodiment, T cells or primary NK cells are expanded on an automated platform and may be transfected by micro-flow through electroporation as described in U.S. Pat. No. 11,377,652, the contents of which are herein incorporated by reference.

T cells may be purified from a tumor, and as such are tumor infiltrating lymphocytes (TIL). TIL may be purified from tumor tissue and expanded ex-vivo. TIL may be reintroduced to the patient as autologous cells or may be administered to a different subject as donor cells. It is anticipated that the stimulation of MHC-I expression by NK cells will lead to enhanced cytotoxic efficacy by TIL.

In an embodiment of the invention, TIL are isolated from a patient tumor by standard techniques. TIL are then exposed to patient tumor tissue, wherein the patient has been treated with NK cells, thereby inducing expression of MHC-I. The NK exposure may be by IV injection, or by intratumoral injection. NK exposure to tumor tissue may be performed ex vivo. TIL exposed to tumor tissue ex vivo are thereby activated and expanded. Expanded TIL comprising CD4 and CD8 cytotoxic T cells are administered to the patient.

T cells may be transfected to express one or more CARs. Transfection techniques include, but are not limited to viral transduction, mRNA transfection, and the Sleeping Beauty transposon system. Subsequent to transfection, CAR T cells may be expanded in a bioreactor until a clinically effective number of cells is obtained.

However, it should be appreciated that in other aspects, the cytotoxic cell may also be a macrophage, a monocyte, a neutrophil cell, a basophile, or cosinophil cell. Therefore, and viewed from a different perspective, the cells contemplated herein may effect cytotoxic action via phagocytosis, pore formation, induction of antibody-dependent cell-mediated cytotoxicity (ADCC), by triggering TNF or fas mediated killing pathways, etc.

Cytotoxic cells may release various types of cytotoxic granules (e.g., granulysin, perforin, granzymes) as part of the cytotoxic anti-tumor process. A variety of assays are available for monitoring cell-mediated cytotoxicity, including flow cytometric assays, e.g., based on presence of lytic granules such as perforin, granzymes, or production of TNF family members, e.g., TNF-α, FasL or TRAIL (Zaritskaya 2010, Clay, T. et al., Clin. Cancer Res. (2001) &: 1127-1135).

In one embodiment, a bodily fluid is obtained after treatment with NK cells, wherein the bodily fluid comprises cellular components, e.g., tumorigenic or cancer cells displaying an antigen to which the CAR-expressing cytotoxic cells described herein bind to, and cytotoxic cells expressing the antigen binding moiety are contacted with the cells. Assays are then performed to detect immune responses, e.g., indicating that an ADCC response or an ADCP response has been triggered by the patient's own immune cells.

Assays for detecting an immune response are known in the art and are described herein. For example, assays for detecting such a response may detect a release of cytotoxic granules (e.g., granulysin, perforin, granzymes), or phagocytosis, or receptor-ligand mediated cytolysis (e.g., as mediated by the Fas/APO pathway). A variety of flow cytometric assays are available for monitoring cell-mediated cytotoxicity, e.g., based on presence of lytic granules such as perforin, granzymes, or production of TNF family members, e.g., TNF-α, FasL or TRAIL (Zaritskaya 2010, Clay, T. et al., Clin. Cancer Res. (2001) &: 1127-1135).

In other embodiments, immune stimulatory cytokines are administered to a patient in combination with the cytotoxic cell (expressing a CAR-based therapeutic (e.g., an antigen binding domain coupled to a CAR scaffold) to promote or trigger an immune response. Cytokines include but are not limited to IL2, IL4, IL7, IL11, IL15, IL21, TNF-alpha, IFN-gamma, etc. In some embodiments, cytokines can reactivate exhausted T cells. In other cases, immune competent cells may be engineered to recombinantly express one or more cytokines.

Other techniques to treat cancer include surgery, radiation therapy, chemotherapy, immunosuppressive reagents (e.g., azathioprine, cyclosporin, methotrexate, mycophenolate, etc.), immunotherapy, targeted therapy, hormone therapy, stem cell transplant, or other precision methods. Any of these techniques may be combined with embodiments of the present invention to treat cancer.

It is understood that present invention embodiments may be administered to a patient using appropriate formulations, indications, and dosing regimens suitable by government regulatory authorities such as the Food and Drug Administration (FDA) in the United States.

In some embodiments, a cytotoxic cell expressing a TCR, a modified TCR, or a CAR-based therapeutic (e.g., an antigen binding domain coupled to a CAR scaffold) is administered to a patient as a pharmaceutical composition. In another embodiment, a method of treating cancer by administration of the cytotoxic cell to a subject is contemplated. In still another embodiment, a method inhibiting the proliferation or reducing the proliferation of a cell that is expressing the corresponding antigen (to which the antigen binding region binds to) on the surface of its cell by administration of the cytotoxic cell to a subject is contemplated.

In an embodiment, the patient may be lymphodepleted, whereby endogenous lymphocyte numbers are reduced, thereby increasing the availability of essential endogenous cytokines and promoting infused T cell survival.

In some embodiments, the cytotoxic cell expressing a TCR, a modified TCR, or a CAR-based therapeutic (e.g., an antigen binding domain coupled to a CAR scaffold) reduces the amount (e.g., number of cells, size of mass, etc.) by at least 25%, at least 50%, at least 75%, at least 90%, at least 95%, or at least 99% in a subject with cancer associated with expression of the corresponding antigen on the surface of the cells relative to a negative control.

Examples of cancer that are treatable by the cytotoxic cells contemplated herein include any cancer expressing or overexpressing a cancer-associated antigen on its cell surface. Examples of cancer that can be treated with a cytotoxic cell expressing a TCR, a modified TCR, or a CAR-based therapeutic (e.g., an antigen binding domain coupled to a CAR scaffold) include but are not limited to breast cancer, colon cancer, leukemia, lung cancer, melanoma, neuroblastoma, pancreatic cancer, pediatric intracranial ependymoma, and prostate cancer.

Dendritic cells (DCs) refer to a diverse population of morphologically similar cell types found in a variety of lymphoid and non-lymphoid tissues, Steinman (1991) Ann. Rev. Immunol. 9:271-296. Dendritic cells constitute the most potent and preferred antigen presenting cells (APCs) in the organism. Dendritic cells can be differentiated from monocytes and possess a distinct phenotype from monocytes. Mature DCs can provide all the signals necessary for T cell activation and proliferation. Also, mature dendritic cells are not phagocytic, whereas the monocytes and immature dendritic cells are strongly phagocytosing cells. Immature DCs are capable of capturing antigens by endocytosis, phagocytosis, macropinocytosis or adsorptive pinocytosis and receptor mediated antigen uptake, and have high intracellular concentrations of MHC class II molecules.

For example, dendritic cells may be obtained from immune competent cells of a patient diagnosed with a cancer are isolated (immune competent cells are typically obtained from the patient apheresis). Alternatively, dendritic cells may also be derived from progenitor cells in response to specific growth factors (e.g., GM-CSF). Regardless of the type of isolation, it is then contemplated that the dendritic cells are transfected with one or more tumor-related epitopes of the tumor of the patient or with an expression vector (preferably viral vector) that includes a nucleic acid that encodes the one or more tumor-related epitopes of the tumor of the patient. Most preferably, the tumor-related epitopes include or are neoepitopes specific to the patient's tumor. As a result, the so transfected dendritic cells will present the tumor epitopes via the MHC-I and/or MHC-II system.

The antigens may be exposed to DCs either naked or in a viral vector (such as an adenoviral vector). Adenovirus vectors are particularly preferred. Moreover, it is further generally preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 February; 72(2): 926-933). Most typically, the desired nucleic acid sequences (for expression from virus infected cells) are under the control of appropriate regulatory elements well known in the art. A viral vector will provide multiple benefits for triggering a strong and durable immune response against the cancer-associated sequences. First, and upon infection of a dendritic cell with the recombinant virus, the cancer-associated sequences are expressed and presented using MHC-I and/or MHC-II presentation pathways, which will increase the likelihood of producing appropriately activated CD4⁺ and CD8⁺ cells, which in turn is believed to increase the likelihood of proper antibody production and suitable T- and B-cell memory.

Thus, the dendritic cells obtained from apheresis of a patient having a tumor are exposed ex vivo to one or more tumor-related epitopes of the tumor of the patient, or to a nucleic acid that encodes one or more tumor related or tumor specific epitopes of the tumor of the patient. In such manner, the immune response can be specifically directed to a particular tumor (and even tumor sub-population), and the immune competent cells of the patient will not be subject to rejection.

Pharmaceutical compositions may comprise cytotoxic cells comprising an antigen binding domain coupled to or linked to a CAR scaffold, as described herein, in combination with one or more pharmaceutically or physiologically acceptable carriers, diluents or excipients. Additionally, pharmaceutical compositions may comprise one or more adjuvants (e.g., aluminum hydroxide), antioxidants, bacteriostats, buffers, carbohydrates, chelating agents such as EDTA or glutathione; coloring, flavoring and/or aromatic substances, emulsifiers, excipients, lubricants, pH buffering agents, preservatives, salts for influencing osmotic pressure, polypeptides (e.g., glycine), proteins, solubilizers, stabilizers, wetting agents, etc., which do not deleteriously react with the active compounds (e.g., antigen binding domain coupled to a CAR scaffold, etc.) or otherwise interfere with their activity. Buffers include but are not limited to neutral buffered saline, phosphate buffered saline, etc. Carbohydrates include but are not limited to dextrans, glucose, mannose, mannitol, sucrose, etc.

Pharmaceutical compositions may be formulated for a particular mode of administration. Modes of administration may include but are not limited to: intraarticular, intradermal, intranasal, intraperitoneal, intrathecally, intratumoral, intravenous, intraventricularly, subcutaneous, transdermal, transmucosal or topical routes.

In preferred embodiments, the cytotoxic cells are administered by intravenous infusion. Such formulations may be prepared according to standard techniques known by one of ordinary skill in the art. For example, a composition that is to be administered intravenously may have one or more ingredients (e.g., a diluent, a suspension buffer, saline or dextrose/water, other components such as cytokines, etc.) prior to infusion in the patient.

Many such techniques for formulating and administering pharmaceutical compositions are known in the art, e.g., U.S. Patent Application Publication No. 2014/0242025, and all such references are incorporated by reference herein in their entirety.

In some embodiments, the cytotoxic cells proliferate in vivo, thereby persisting in the patient for months or even years after administration to provide a sustained mechanism for inhibiting tumor growth or recurrence. In some aspects, the cytotoxic cells persist at least for three months, six months, nine months, twelve months, fifteen months, eighteen months, two years, three years, four years, or five years after administration of the cytotoxic cells to the patient.

Cytotoxic cells may be obtained from any of a variety of sources, (e.g., isolated from a human, from commercially available cytotoxic cells, from a cell repository, etc.). Procedures for ex vivo expansion of NK cells, T cells or other types of cytotoxic cells are known in the art (e.g., Smith et al., Clinical & Translational Immunology (2015) 4: e31). The examples presented herein are not intended to be limited to any particular method of ex vivo expansion of cytotoxic cells.

Pharmaceutical compositions comprising cytotoxic cells, as described herein, may be administered at a dosage of 10⁴ to 10⁹ cells/kg body weight, of 10⁵ to 10⁶ cells/kg body weight, or any integer values within these ranges. Cytotoxic cell compositions may be administered one time or serially (over the course of days or weeks or months) at these dosages. Infusion techniques for cytotoxic cells, such as T cells, are known in the art (see, e.g., Rosenberg et al., New Eng. J. of Med. 319: 1676, 1988).

In other embodiments, the pharmaceutical compositions are administered in a therapeutically effective amount, which is the amount effective for treating the specific indication. Administration may occur as a one-time dose or based on an interval. As used herein, “interval” indicates that the therapeutically effective amount is administered periodically (as distinguished from a one-time dose). The administration interval for a single individual need not occur at a fixed interval but can vary over time. The term, “in combination with” or “co-administered” indicates that a composition can be administered shortly before, at or about the same time, or shortly after another composition.

### EXAMPLES

Example 1: In one example disclosed herein, tumor targeted lymphocytes are generated for use in the treatment of a cancer or infectious disease. The method comprises the steps of performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction, wherein the remaining apheresis product comprises a CD3−/CD14− fraction; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; and purifying the expanded T cells.

The T cells may be expanded in the presence of IL-15 or an agonist derivative thereof. Furthermore, the T cells may be genetically modified to express endoplasmic reticulum localized IL-15 (erIL-15) and/or to express a chimeric antigen receptor (CAR) wherein the CAR targets a tumor antigen or a checkpoint inhibitor. The adenovirus disclosed herein may be an Ad5 adenovirus. Moreover, the dendritic cells may be further exposed to a modified RNA and/or a lentivirus and/or a peptide pool of neoepitopes.

Example 2: In another example disclosed herein, tumor targeted natural killer (NK) cells are generated for use in the treatment of a cancer or infectious disease. The method comprises the steps of performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from the CD3− CD14− fraction of the apheresis; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; purifying the expanded T cells; isolating from the expanded T cells at least one nucleic acid encoding an α and β chain of a T cell receptor (TCR), and fusing the nucleic acid to the 5′ end of a second nucleic acid encoding a transmembrane domain and an intracellular signaling domain of a chimeric antigen receptor (CAR), wherein the fused nucleic acid encodes a TCR CAR; and transfecting the enriched and expanded NK cells with a nucleic acid encoding the TCR CAR.

The NK cells disclosed herein may comprise NK-92 cells or memory cytokine enriched NK cells (M-CENK). Furthermore, the M-CENK cells may be genetically modified to express CD16 and/or a CAR that targets a tumor antigen or a checkpoint inhibitor. Moreover, the NK-92 cells may comprise CD16 and/or they may be further genetically modified to express a second CAR.

Example 3: In yet another example, the inventors have disclosed a method of expanding tumor infiltrating lymphocytes for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from the CD3−/CD14− fraction of the apheresis; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; purifying the expanded T cells; purifying CD3+ TIL from a solid tumor and exposing the TIL to the activated DC, thereby expanding the TIL; and purifying the expanded TIL.

Example 4: In a further example, a pharmaceutical composition is disclosed. The pharmaceutical composition comprises 1) activated dendritic cells (DC), wherein the DC are differentiated from patient apheresis-derived CD14+ monocytes; 2) an adenovirus encoding an antigenic peptide sequence, wherein the DC are activated upon exposure to the adenovirus; and 3) patient apheresis-derived CD3+ T cells, wherein the CD3+ T cells are exposed to the activated DC, thereby activating and expanding the T cells. It is contemplated that the pharmaceutical composition is used in the treatment of a cancer or infectious disease.

In the pharmaceutical composition disclosed above, the DC MHC-I or MHC-II present the antigenic peptide sequence or a portion thereof, thereby activating the DC. The activated CD3+ T cells may comprise a T cell receptor (TCR) with specificity for the MHC-presented antigenic peptide sequence on the DC.

The pharmaceutical composition may further comprise Natural Killer (NK) cells. In some instances, the NK cells are expanded from the patient apheresis. The NK cells may also comprise cytokine enriched (CENK) cells or memory-like cytokine enriched (M-CENK) cells. The NK cells are expanded in a medium comprising IL-15, IL12, and/or IL-18. In some embodiments, the NK cells are NK-92 cells, and more preferably NK-92 cells that comprise a high affinity Fc receptor and/or an endoplasmic reticulum targeted IL-2 (erIL-2) or an erIL-15. The NK cells may also comprise a CAR. It is contemplated that the CAR comprises a targeting domain, and wherein the targeting domain comprises α and β chains of a TCR. Preferably, the αand β chains are from the patient's activated T cells. In some embodiments, the CAR comprises a targeting domain, and wherein the targeting domain comprises an antibody binding domain. Preferably, the antibody binding domain is specific for a tumor associated antigen, a tumor specific antigen, or a neoepitope. Alternatively, or additionally, the antibody binding domain is specific for a checkpoint inhibitor.

It is further contemplated that the pharmaceutical composition comprises IL-15 or an agonist derivative thereof, wherein the T cells are exposed to IL-15 or an agonist derivative thereof prior to exposure to activated DC and/or during exposure to activated DC. The dendritic cells of the pharmaceutical composition disclosed herein are preferably activated in a medium comprising GM-CSF (Granulocyte-macrophage colony-stimulating factor). Furthermore, the dendritic cells may be activated in a medium further comprising IL-4.

Example 5: In another example, the inventors have disclosed a pharmaceutical composition comprising patient-derived dendritic cells (DC), GM-CSF, and an adenovirus (Ad) encoding an antigenic peptide, for use in the treatment of a cancer or infectious disease, wherein the DC are derived from the patient's apheresis. The pharmaceutical composition may further comprise IL-4 and/or IL-15. The MOI (Multiplicity of Infection) of the pharmaceutical composition is contemplated to be in the range of 20-20,000.

Example 6: In yet another example, the inventors have disclosed a pharmaceutical composition comprising 1) patient-derived Natural Killer (NK) cells and/or Natural Killer T (NKT) cells, 2) GM-CSF, and 3) an adenovirus (Ad) encoding an antigenic peptide, for use in the treatment of a cancer or infectious disease, wherein the NK or NKT cells are derived from the patient's apheresis. The pharmaceutical composition may further comprise IL-4 and/or IL-15. The MOI (Multiplicity of Infection) of the pharmaceutical composition is contemplated to be in the range of 20-20,000.

Example 7: A pharmaceutical composition comprising 1) patient-derived T cells, B cells, and/or monocytes, 2) GM-CSF, and 3) an adenovirus (Ad) encoding an antigenic peptide, for use in the treatment of a cancer or infectious disease, wherein the T cells, B cells, and/or monocytes are derived from the patient's apheresis. The pharmaceutical composition may further comprise IL-4 and/or IL-15. The MOI (Multiplicity of Infection) of the pharmaceutical composition is contemplated to be in the range of 20-20,000.

Example 8: A pharmaceutical composition comprising 1) dendritic cells (DC), 2) an irradiated biopsy sample, and 3) T-cells, wherein the DC and T-cells are derived from apheresis of a patient, and the biopsy sample is from a tumor of the same patient, and wherein the pharmaceutical composition is for use in the treatment of a cancer.

The pharmaceutical composition may further comprise IL-4 and/or IL-15. The MOI (Multiplicity of Infection) of the pharmaceutical composition is contemplated to be in the range of 20-20,000. The pharmaceutical composition may also further comprise an adenovirus (Ad), wherein the Ad comprises a nucleic acid encoding an antigenic peptide sequence.

The pharmaceutical composition may further comprise Natural Killer (NK) cells. In some instances, the NK cells are expanded from the patient apheresis or the patient PBMC. The NK cells may also comprise cytokine enriched (CENK) cells or memory-like cytokine enriched (M-CENK) cells. The NK cells are expanded in a medium comprising IL-15, IL12, and/or IL-18. In some embodiments, the NK cells are NK-92 cells, and more preferably NK-92 cells that comprise a high affinity Fc receptor and/or an endoplasmic reticulum targeted IL-2 (erIL-2) or an erIL-15. The NK cells may also comprise a CAR. It is contemplated that the CAR comprises a targeting domain, and wherein the targeting domain comprises α and β chains of a TCR. Preferably, the α and β chains are from the patient's activated T cells. In some embodiments, the CAR comprises a targeting domain, and wherein the targeting domain comprises an antibody binding domain. Preferably, the antibody binding domain is specific for a tumor associated antigen, a tumor specific antigen, or a neoepitope. Alternatively, or additionally, the antibody binding domain is specific for a checkpoint inhibitor.

In some instances, the biopsy sample and the T-cells are sequentially added to the DC. For example, the biopsy sample may be first combined with the DC, and then the T-cells are added, wherein the T-cells are expanded T cells.

Example 9: In another example, the inventors have disclosed a pharmaceutical composition comprising dendritic cells (DC) and T-cells, wherein the DC and T-cells are isolated from apheresis of a patient, wherein the DC and/or T-cells are exposed to a biopsy sample from a tumor of the same patient, and wherein the pharmaceutical composition is formulated for administration to the patient.

Preferably, the DC are further exposed to an adenovirus (Ad), wherein the Ad comprises a nucleic acid encoding an antigenic peptide sequence. The DC and/or T-cells may also be further exposed to IL-15, or an agonist derivative thereof. The biopsy sample is ideally from a tumor of the patient who has been treated with a DAMP inducer, wherein the DAMP inducer comprises radiation and/or a Histone Deacetylase (HDAC) inhibitor. The DC are exposed the biopsy in the presence of an activation medium comprising and granulocyte colony stimulating factor (GMCSF) and IL-4, wherein the DC are activated and matured. The exposure of the DC and biopsy to activation medium may be for 24-48 hr, and wherein the DC are then isolated and combined with the T-cells. The pharmaceutical composition may be formulated for administration intravenously, subcutaneously, intratumorally, or by instillation.

Example 10: In another example, the inventors have disclosed a pharmaceutical composition comprising dendritic cells (DC) and T-cells, wherein an apheresis sample from a patient is exposed to a biopsy sample from a tumor of the same patient, wherein the DC and T-cells are then isolated from the apheresis, and wherein the pharmaceutical composition is formulated for administration to the patient.

Preferably, the DC are further exposed to an adenovirus (Ad), wherein the Ad comprises a nucleic acid encoding an antigenic peptide sequence. The DC and/or T-cells may also be further exposed to IL-15, or an agonist derivative thereof. The isolated DC may be further exposed to an adenovirus (Ad), wherein the Ad comprises a nucleic acid encoding an antigenic peptide sequence. The biopsy sample is ideally from a tumor of the patient who has been treated with a DAMP inducer, wherein the DAMP inducer comprises radiation and/or a Histone Deacetylase (HDAC) inhibitor. The DC and/or CD14+ Monocytes are exposed to the biopsy in the presence of an activation medium comprising and granulocyte colony stimulating factor (GMCSF) and IL-4, wherein the DC are activated and matured. The exposure of the DC and/or CD14+ Monocytes and biopsy to activation medium may be for 24-48 hr, and wherein the differentiated DCs are then isolated and combined with the purified CD3+ T-cells. The pharmaceutical composition may be formulated for administration intravenously, subcutaneously, intratumorally, or by instillation.

Example 11: In another example, the inventors have disclosed a method of generating tumor targeted lymphocytes for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from CD3−/CD14− fraction of the apheresis; differentiating the CD14+ Monocytes into dendritic cells (DC), and exposing to DC to at least one compound selected from the group consisting of a protein arginine methyltransferase 5 (PRMT5) inhibitor, a DNA methyltransferase (DNMT) inhibitor, and a histone deacetylase (HDAC) inhibitor, whereby the DC MHC-I or MHC-II present at least one re-expressed peptide sequence or a portion thereof, thereby activating the DC; and exposing the purified CD3+ T cells to the activated DC, thereby expanding the T cells;

Example 12: In yet another example, the inventors have disclosed a method of generating tumor targeted natural killer (NK) cell for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from CD3−/CD14− fraction of the apheresis; differentiating the CD14+ Monocytes into dendritic cells (DC), and exposing to DC to at least one compound selected from the group consisting of a protein arginine methyltransferase 5 (PRMT5) inhibitor, a DNA methyltransferase (DNMT) inhibitor, and a histone deacetylase (HDAC) inhibitor, whereby the DC MHC-I or MHC-II present at least one re-expressed peptide sequence or a portion thereof, thereby activating the DC; exposing the purified CD3+ T cells to the activated DC, thereby expanding the T cells; isolating from the expanded T cells at least one nucleic acid encoding an α and β chain of a T cell receptor (TCR), and fusing the nucleic acid to the 5′ end of a second nucleic acid encoding a transmembrane domain and an intracellular signaling domain of a chimeric antigen receptor (CAR), wherein the fused nucleic acid encodes a TCR CAR; transfecting the enriched and expanded NK cells with a nucleic acid encoding the TCR CAR.

Example 13: In this example the inventors have disclosed a method of expanding tumor infiltrating lymphocytes for use in the treatment of a cancer or infectious disease. The method comprises performing therapeutic apheresis on a subject with cancer or infectious disease; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; expanding NK cells from the CD3−/CD14− fraction of the apheresis; differentiating the CD14+ Monocytes into dendritic cells (DC), and exposing to DC to at least one compound selected from the group consisting of a protein arginine methyltransferase 5 (PRMT5) inhibitor, a DNA methyltransferase (DNMT) inhibitor, and a histone deacetylase (HDAC) inhibitor, whereby the DC MHC-I or MHC-II present at least one re-expressed peptide sequence or a portion thereof, thereby activating the DC; purifying CD3⁺ TIL from a solid tumor and exposing the CD3+ TIL to the activated DC, thereby expanding the TIL; and purifying the expanded TIL.

Example 14: A pharmaceutical composition comprising tumor targeted CD3⁺ T lymphocytes is disclosed herein, wherein the pharmaceutical composition is for use in the treatment of a cancer or infectious disease. The composition includes 1) dendritic cells (DC), wherein the DC are differentiated from patient-derived apheresis purified CD14+ monocytes, 2) at least one compound selected from the group consisting of a protein arginine methyltransferase 5 (PRMT5) inhibitor, a DNA methyltransferase (DNMT) inhibitor and a histone deacetylase (HDAC) inhibitor, whereby the DC MHC-I or MHC-II present at least one re-expressed peptide sequence or a portion thereof, thereby activating the DC, and 3) CD3+ T cells purified from the patient apheresis; wherein the T cells are exposed to the activated DC, thereby activating and expanding the T cells.

Example 15: In another example, the inventors have disclosed a method of expanding tumor infiltrating lymphocytes for use in the treatment of a cancer or infectious disease. The method comprises performing therapeutic apheresis on a subject with cancer or infectious disease, wherein the subject has been treated with at least one therapeutic agent selected from the group consisting of a protein arginine methyltransferase 5 (PRMT5) inhibitor, a DNA methyltransferase (DNMT) inhibitor, and a histone deacetylase (HDAC) inhibitor; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; purifying CD3⁺ TIL from a solid tumor and exposing the TIL to the activated DC, thereby expanding the TIL; purifying the expanded TIL.

Example 16: The inventors have also disclosed herein a method of generating tumor targeted lymphocytes for use in the treatment of a cancer or infectious disease. The method comprises performing therapeutic apheresis on a subject with cancer or infectious disease, wherein the subject has been treated with at least one therapeutic agent selected from the group consisting of a protein arginine methyltransferase 5 (PRMT5) inhibitor, a DNA methyltransferase (DNMT) inhibitor and a histone deacetylase (HDAC) inhibitor; purifying a CD3+ T cell fraction from the apheresis product, wherein the remaining apheresis product comprises a CD3− fraction; purifying CD14+ Monocytic cells from the CD3− fraction of the apheresis, wherein the remaining apheresis product comprises a CD3−/CD14− fraction; differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC; exposing the purified CD3+ T cells to the activated DC, thereby expanding the T cells; purifying the expanded T cells.

Example 17:

In another example, the inventors have disclosed a method of transduction of fresh PBMCs with adenovirus control AD5-[E1-, E2b-]-GFP. Fresh blood from 2 donors were used for PBMC isolation and transduction of Ad5-GFP. Cells were infected at MOI of 20. Post transduction, the cells were cultured in AIM-V medium containing either N-803 or GM-CSF, or GM-CSF + IL-4. GFP expression was monitored using IncuCyte and evaluated using flow-cytometry. The following conditions were evaluated:

TABLE 1

| Conditions | N-803 | GM-CSF | GM-CSF + IL-4 |
|---|---|---|---|
| 1 | + | | |
| 2 | | + | |
| 3 | | | + |

The MOI used in the experiments above was 20. The transduction details are as follows: 12-well plates, for Donor 1 used 1×10⁶ cells/well and for donor 2 used 5×10⁵ cells per well. The following quantities of N-803, GM-CSF & IL-4 were used: 74 ng/ml N-803, 100 ng/mL GM-CSF & 20 ng/mL IL-4. The virus used for the transduction was Ad5-[E1−, E2b−]-GFP.

The transduction was performed in 12-well plates. Isolated PBMCs were used for transduction in AIM-V medium (250 μL). Ad5 virus was added at MOI 20 (250 μL). Virus infected culture was placed at 37° C., 5% CO₂ incubator for 1hour. 0.5 mL of media containing the corresponding cytokines were added after 1 hour and the plates were incubated for additional 6 days (Note: plates left for longer to get more cells to perform staining for flow cytometry). GFP expression was monitored using IncuCyte and evaluated using flow cytometry

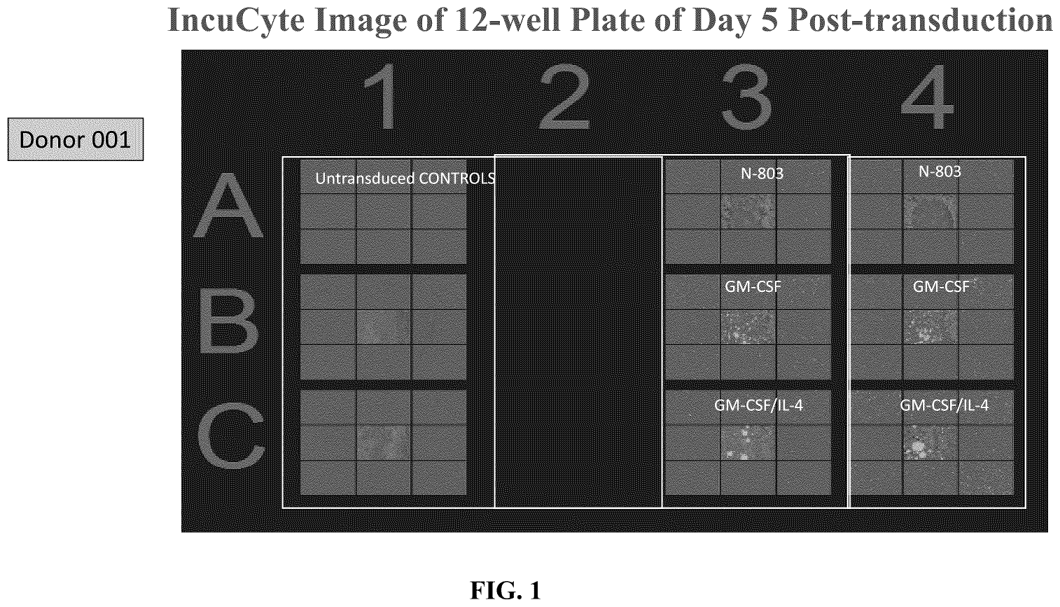

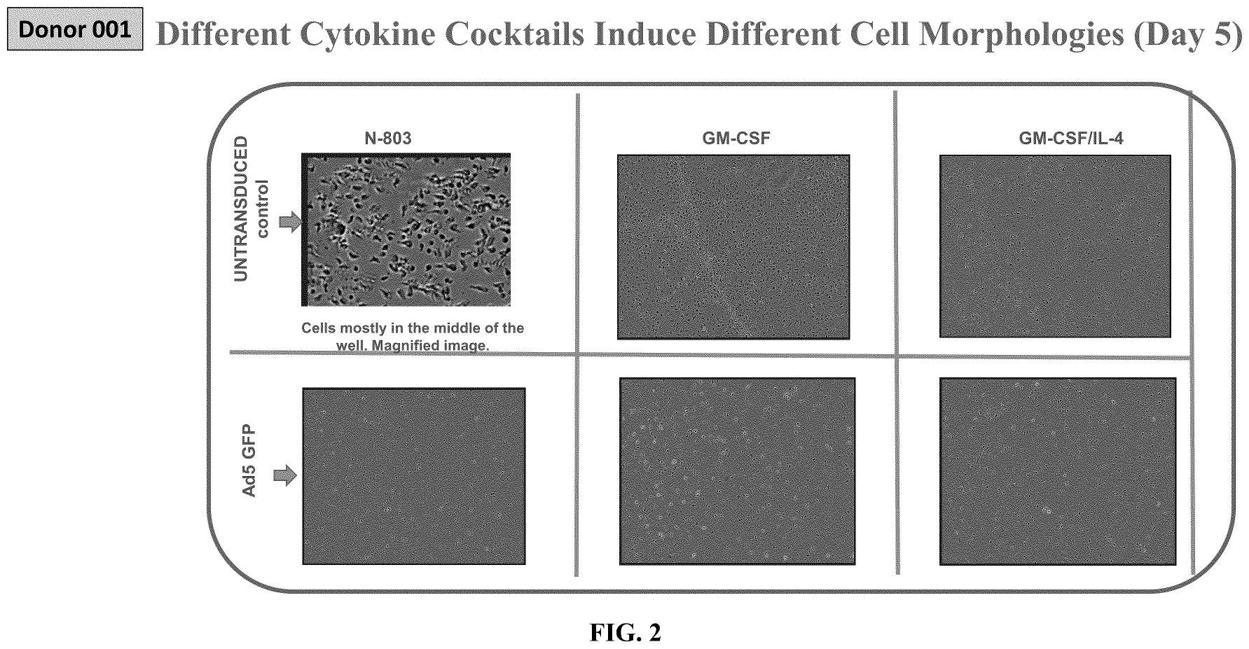

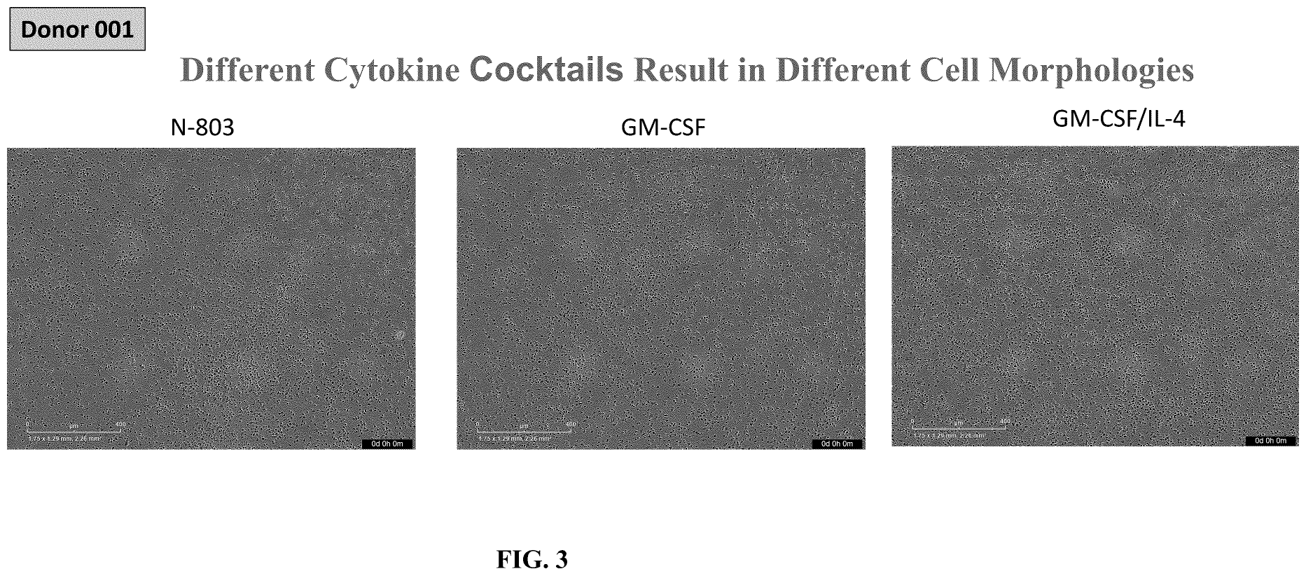

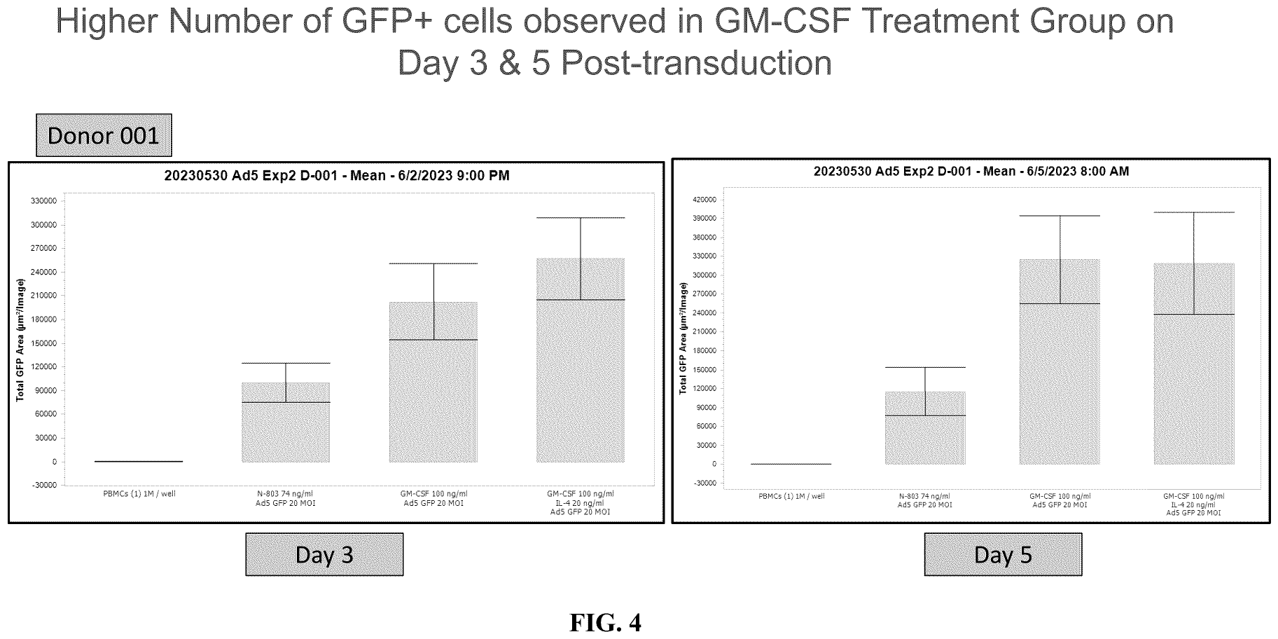

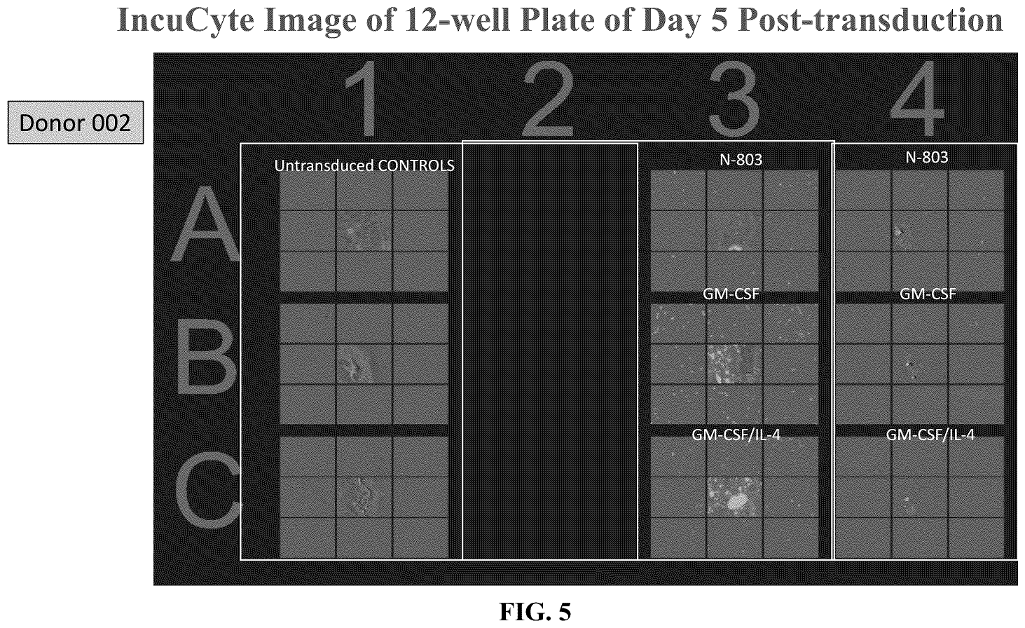

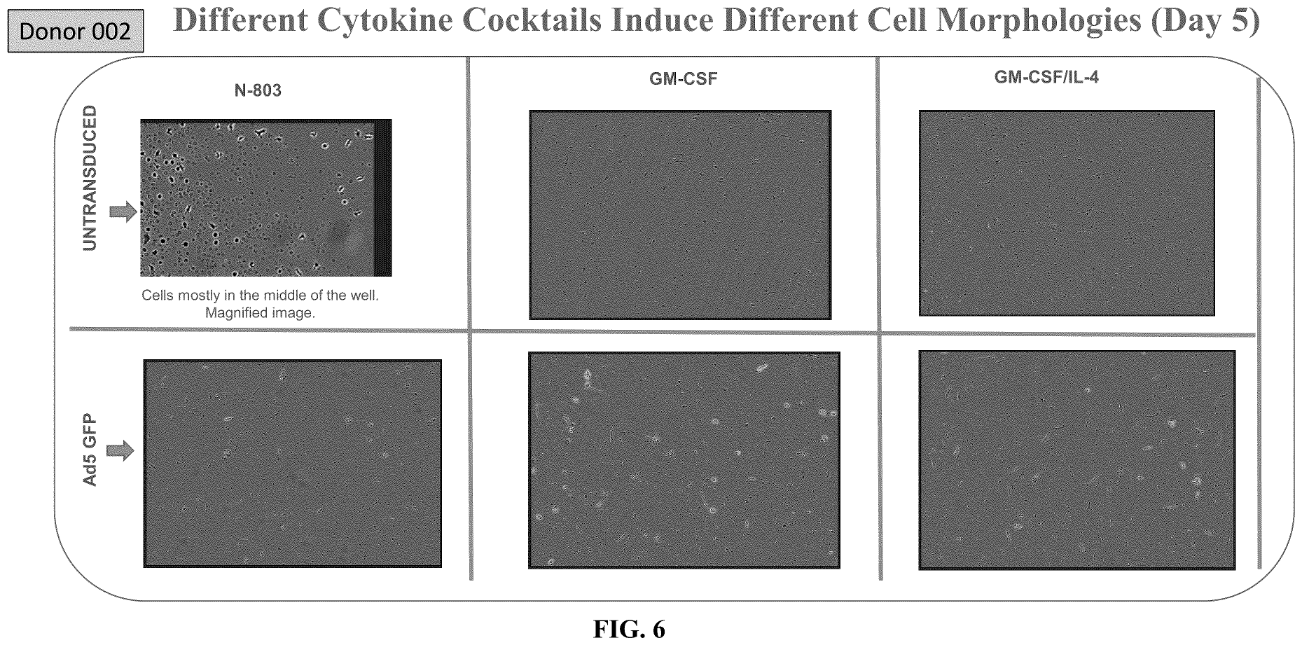

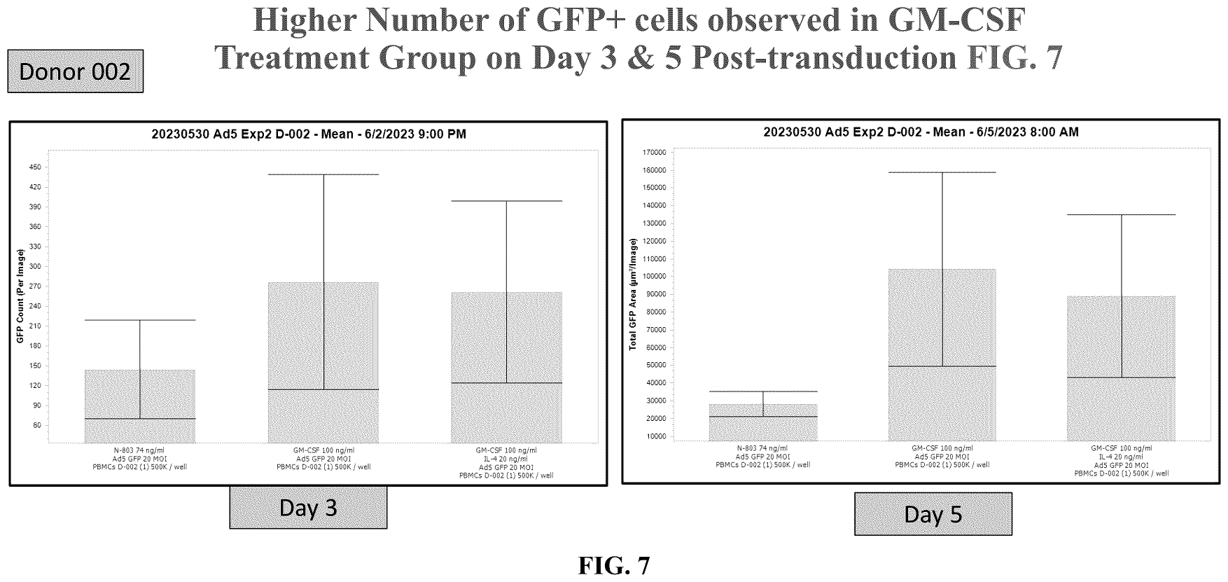

The results are shown in FIGS. 1-8 for two donors (donor 001 and donor 002). FIGS. 1-3 and 5-6 show that different cytokine cocktails (N-803, GM-CSF & IL-4) induce different cell morphologies in donor 001 and donor 002 respectively. FIGS. 4 & 7 shows that higher number of GFP+ cells were observed in GM-CSF Treatment Group on Day 3 & 5 Post-transduction for donor 001 and donor 2 respectively.

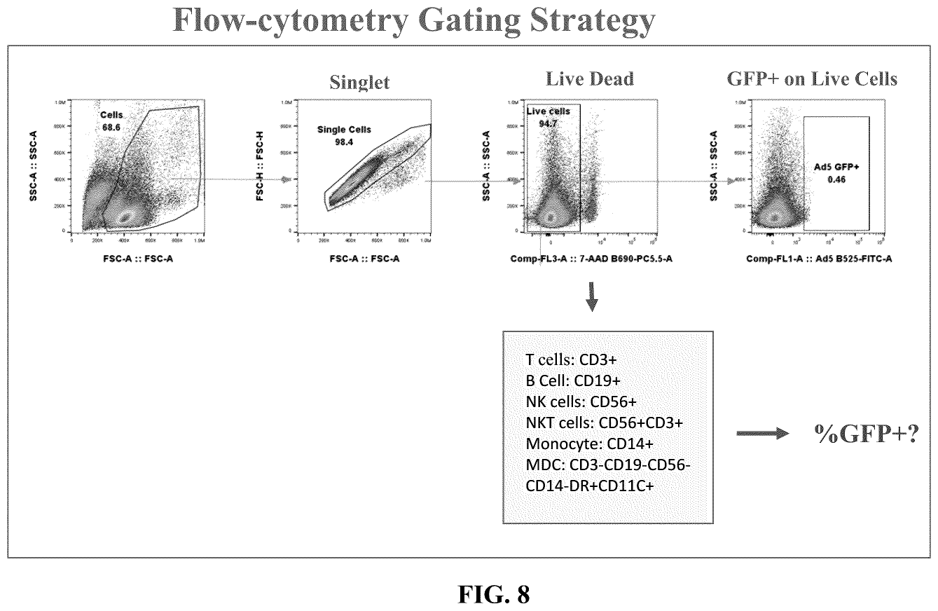

The flow cytometry gating strategy is shown in FIG. 8, and the results are also presented in tabular format below. The two tables below (Tables 2-3) show calculated % GFP+ cells on Day 6 by Flow Cytometry

TABLE 2
Donor 1, Day 6

| | | | | GFP+ Cells of | | |
|---|---|---|---|---|---|---|
| | % Population | | | Percent population | | |
| | | | GM-CSF + | | | GM-CSF + |
| | N-803 | GM-CSF | IL-4 | N-803 | GM-CSF | IL-4 |
| T cell | 60.7 | 57.7 | 59.2 | 1.96 | 10.7 | 10.6 |
| B Cell | 2.12 | 4.16 | 3.35 | 0.8 | 2.62 | 5.9 |
| NK cell | 3.10 | 5.92 | 5.58 | 4.71 | 7.89 | 7.33 |
| NKT cells | 11.6 | 7.5 | 7.09 | 4.91 | 19.6 | 15.9 |
| Monocytes | 0.2 | 0.22 | 0.19 | 10.1 | 18.2 | 33 |
| MDC | 0.02 | 0.01 | 0.11 | 33.3 | 50 | 38.9 |

TABLE 3
Donor 2, day 6

| | | | | GFP+ cells of | | |
|---|---|---|---|---|---|---|
| | % Population | | | percent population | | |
| | | | GM-CSF + | | | GM-CSF + |
| | N-803 | GM-CSF | IL-4 | N-803 | GM-CSF | IL-4 |
| T cell | 82.8 | 71.1 | 69.2 | 1.94 | 1.56 | 1.25 |
| B Cell | 2.35 | 3.35 | 3.37 | 0.16 | 0.56 | 0.42 |
| NK cell | 2.34 | 7.13 | 3.99 | 2.38 | 1.78 | 2.71 |
| NKT cells | 23.7 | 10.7 | 9.51 | 7.22 | 6.94 | 4.23 |
| Monocytes | 0.28 | .37 | 0.19 | 5.92 | 6.10 | 11.2 |
| MDC | 0.033 | .27 | 0.48 | 100 | 10.5 | 21.1 |

The % GFP+ cells on Day 23 by Flow Cytometry is shown in tables 4-5 below

TABLE 4
Donor 1, day 23

| | | | GFP + cells of percent | |
|---|---|---|---|---|
| | % Population | | population | |
| | | GM-CSF + | | GM-CSF + |
| | GM-CSF | IL-4 | GM-CSF | IL-4 |
| T cell | 92.2 | 97.6 | 14.0 | 5.79 |
| B Cell | 0.022 | 0.003 | 0 | 0 |
| NK cell | 5.37 | 0.56 | 4.59 | 0.55 |
| NKT cells | 27.1 | 5.43 | 24.6 | 38.4 |
| Monocytes | 0.26 | 0.17 | 8.57 | 12.3 |
| MDC | 2.22 | 0 | 50.0 | 0 |

TABLE 5
Donor 2, day 23

| | | | GFP + cells of percent | |
|---|---|---|---|---|
| | % Population | | population | |
| | | GM-CSF + | | GM-CSF + |
| | GM-CSF | IL-4 | GM-CSF | IL-4 |
| T cell | 92.2 | 97.6 | 14.0 | 5.79 |
| B Cell | 0.022 | 0.003 | 0 | 0 |
| NK cell | 5.37 | 0.56 | 4.59 | 0.55 |
| NKT cells | 27.1 | 5.43 | 24.6 | 38.4 |
| Monocytes | 0.26 | 0.17 | 8.57 | 12.3 |
| MDC | 2.22 | 0 | 50.0 | 0 |

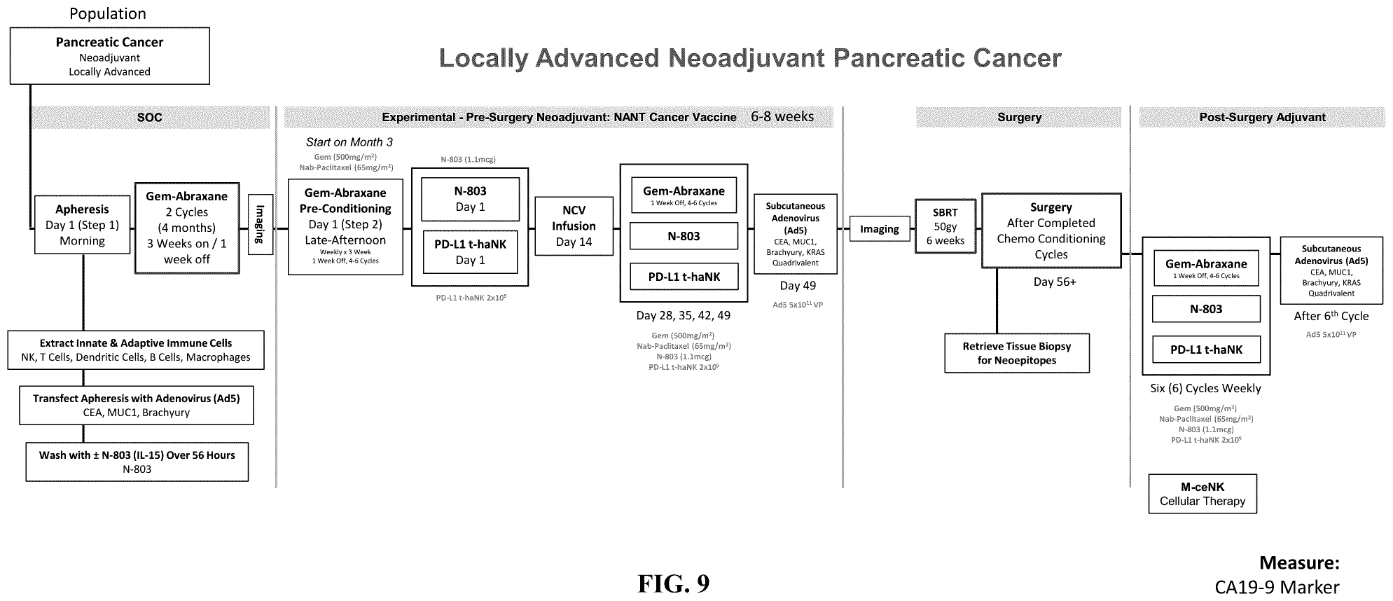

FIG. 9 shows a treatment chart for locally advanced neoadjuvant pancreatic cancer. In this example, therapeutic apheresis is performed prior to treatment with the Nant Cancer Vaccine. Nant Cancer Vaccine is discussed in more detail in US Patent Publication numbers US20190381156A1 and US20190318804A1, which are incorporated by reference in its entirety. The apheresis product is transfected with adenovirus encoding at least one immunogenic peptide sequence as indicated, wherein the peptide sequence comprises a tumor associated antigen or a neoepitope. The transduced apheresis product is stimulated with an immunostimulant. N-803 is indicated in the figure. T cells exposed to the transduced apheresis product are expanded and formulated for administration to the patient before or after surgery. Of course, the incorporation of the therapeutic apheresis product of the invention into a therapeutic regime is not limited to this example.

The above discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the inventive subject matter are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the inventive subject matter are approximations, the numerical values set forth in the specific examples are reported as precisely as practicable. The numerical values presented in some embodiments of the inventive subject matter may contain certain errors necessarily resulting from the standard deviation found in their respective testing measurements.

In some embodiments, the numbers expressing quantities of ingredients, properties such as concentration, reaction conditions, and so forth, used to describe and claim certain embodiments of the inventive subject matter are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the inventive subject matter are approximations, the numerical values set forth in the specific examples are reported as precisely as practicable. The numerical values presented in some embodiments of the inventive subject matter may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the inventive subject matter and does not pose a limitation on the scope of the inventive subject matter otherwise claimed. No language in the specification should be construed as indicating any non-claimed element essential to the practice of the inventive subject matter.

Groupings of alternative elements or embodiments of the inventive subject matter disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

What is claimed is:

1. A method of generating tumor targeted lymphocytes for use in the treatment of a cancer or infectious disease, the method comprising:
i. performing therapeutic apheresis on a subject with cancer or infectious disease;
ii. purifying a CD3+ T cell fraction from the apheresis product, wherein the remaining apheresis product comprises a CD3− fraction;
iii. purifying CD14+ Monocytic cells from the CD3− fraction, wherein the remaining apheresis product comprises a CD3−/CD14− fraction;
iv. differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC;
v. exposing the purified CD3+ T cells to the activated DC, thereby expanding the T cells;
vi. purifying the expanded T cells.

2. The method of claim 1, wherein the T cells are expanded in the presence of IL-15 or an agonist derivative thereof.

3. The method of claim 1, wherein the T cells are genetically modified to express endoplasmic reticulum localized IL-15 (erIL-15).

4. The method of claim 1, wherein the T cells are genetically modified to express a chimeric antigen receptor (CAR), and wherein the CAR targets a tumor antigen or a checkpoint inhibitor.

5. The method of claim 1, wherein the adenovirus comprises an Ad5 adenovirus.

6. The method of claim 1, wherein the dendritic cells are further exposed to a modified RNA.

7. The method of claim 1, wherein the dendritic cells are further exposed to a lentivirus.

8. The method of claim 1, wherein the dendritic cells are further exposed to a peptide pool of neoeptiopes.

9. A method of generating tumor targeted natural killer (NK) cell for use in the treatment of a cancer or infectious disease, the method comprising:
i. performing therapeutic apheresis on a subject with cancer or infectious disease;
ii. purifying a CD3+ T cell fraction from the apheresis product, wherein the remaining apheresis product comprises a CD3− fraction;
iii. purifying CD14+ Monocytic cells from the CD3− fraction of the apheresis, wherein the remaining apheresis product comprises a CD3−/CD14− fraction;
iv. expanding NK cells from the CD3− CD14− fraction of the apheresis;
v. differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC;
vi. exposing the purified CD3+ T cells to the activated DC, thereby expanding the T cells;
vii. purifying the expanded T cells.
viii. isolating from the expanded T cells at least one nucleic acid encoding an α and β chain of a T cell receptor (TCR), and fusing the nucleic acid to the 5′ end of a second nucleic acid encoding a transmembrane domain and an intracellular signaling domain of a chimeric antigen receptor (CAR), wherein the fused nucleic acid encodes a TCR CAR;
ix. transfecting the enriched and expanded NK cells with a nucleic acid encoding the TCR CAR.

10. The method of claim 8, wherein the NK cells comprise memory cytokine enriched NK cells (M-CENK).

11. The method of claim 9 wherein the M-CENK are genetically modified to express CD16.

12. The method of claim 9, wherein the M-CENK cells are further genetically modified to express a CAR, wherein the CAR targets a tumor antigen or a checkpoint inhibitor.

13. The method of claim 12, wherein the NK-92 cells comprise CD16.

14. The method of claim 12, wherein the NK-92 cells are further genetically modified to express a second CAR.

15. A method of expanding tumor infiltrating lymphocytes for use in the treatment of a cancer or infectious disease, the method comprising:
i. performing therapeutic apheresis on a subject with cancer or infectious disease;
ii. purifying a CD3+ T cell fraction from the apheresis product, wherein the remaining apheresis product comprises a CD3− fraction;
iii. purifying CD14+ Monocytic cells from the CD3− fraction of the apheresis, wherein the remaining apheresis product comprises a CD3−/CD14− fraction;
iv. expanding NK cells from the CD3−/CD14− fraction of the apheresis;
v. differentiating the CD14+ Monocytes into dendritic cells (DC), and exposing the DC to an antigenic peptide or an adenovirus encoding an antigenic peptide sequence, wherein the DC MHC-I or MHC-II present the peptide sequence or a portion thereof, thereby activating the DC;
vi. exposing the purified CD3+ T cells to the activated DC, thereby expanding the T cells;
vii. purifying the expanded T cells;
viii. purifying CD3+ TIL from a solid tumor and exposing the TIL to the activated DC, thereby expanding the TIL;
ix. purifying the expanded TIL.

16. A pharmaceutical composition comprising tumor targeted lymphocytes for use in the treatment of a cancer or infectious disease, the composition comprising 1) activated dendritic cells (DC), wherein the DC are differentiated from patient apheresis-derived CD14+ monocytes; 2) an adenovirus encoding an antigenic peptide sequence, wherein the DC are activated upon exposure to the adenovirus; and 3) patient apheresis-derived CD3+ T cells, wherein the CD3+ T cells are exposed to the activated DC, thereby activating and expanding the T cells.

17. The pharmaceutical composition of claim 16, wherein DC MHC-I or MHC-II present the antigenic peptide sequence or a portion thereof, thereby activating the DC.

18. The pharmaceutical composition of claim 16, wherein the activated CD3+ T cells comprise a T cell receptor (TCR) with specificity for the MHC-presented antigenic peptide sequence on the DC.

19. The pharmaceutical composition of claim 16, further comprising Natural Killer (NK) cells.

20. The pharmaceutical composition of claim 19, wherein the NK cells are expanded from the patient apheresis.
